SEQUENCE LISTING (8 sequences)

SEQ ID NO: 1 - protein, 458 aa, homo sapiens
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPKELAKLVNKRSDFASNCCSINSPPLYCDSEIDAELKNIL

SEQ ID NO: 2 - DNA, 1801 bp, homo sapiens
tttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaa

SEQ ID NO: 3 - protein, 458 aa, homo sapiens
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPEATPTELAKLVNKRSDFASNCCSINSPPLYCDSEIDAELKNIL

SEQ ID NO: 4 - protein, 458 aa, homo sapiens
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKRSDFASNCCSINSPPLYCDSEIDAELKNIL

SEQ ID NO: 5 - DNA, 1801 bp, homo sapiens
tttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgacgaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaa

SEQ ID NO: 6 - DNA, 1801 bp, homo sapiens
tttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgaggctgaattgacgaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaa

SEQ ID NO: 7 - DNA, 26 bp, artificial sequence
  /note=“Description of artificial sequence primer”
tgtagtaagaccttacatttaaatgg

SEQ ID NO: 8 - DNA, 25 bp, artificial sequence
  /note=“Description of artificial sequence primer”
tacgttcttaaaagattctgccatg

(19) United States
(12) Patent Application Publication
(10) Pub. No.: US 2012/0190561 A1
(43) Pub. Date: Jul. 26, 2012

(54) MEANS AND METHODS FOR DIAGNOSING ENDOMETRIOSIS

(76) Inventors: Ludwig Wildt, Lans (AT); Beata Seeber, Sterzing (AT); Klaus Faseral, Kolsass (AT); George Golderer, Innsbruck (AT); Leopold Kremser, Innsbruck (AT); Herbert Lindner, Grinzens (AT); Bettina Sarg, Axems (AT)

(21) Appl. No.: 13/295,805

(22) Filed: Nov. 14, 2011

Related U.S. Application Data
(60) Provisional application No. 61/413,770, filed on Nov. 15, 2010.

(51) Int. Cl. C12Q 1/68; C07H 21/04; C07K 16/18; G01N 33/566; C40B 30/00

(52) U.S. Cl. 506/7; 435/7.92; 435/6.12; 530/389.8; 536/24.33; 536/24.31

(57) ABSTRACT

The present invention relates to means and methods for diagnosing or predicting endometriosis in a female subject. Particularly, the present invention relates to methods for determining the susceptibility to, predisposition for, presence of and/or risk of developing or suffering from endometriosis in a female subject. The present invention also relates to a kit useful for determining the risk of developing or suffering from endometriosis in a subject, a binding molecule specifically binding to the DBP GC*2 or DBP GC*1 allele product, and a binding molecule binding to the gene encoding the DBP GC*2 or GC*1 allele.

This application claims priority to U.S. Application No. 61/413,770, filed on Nov. 15, 2010, the entire contents of which is hereby incorporated by reference.

### BACKGROUND OF THE INVENTION

### FIELD OF THE INVENTION

The present invention relates to means and methods for diagnosing or predicting endometriosis in a female subject. Particularly, the present invention relates to methods for determining the susceptibility to, predisposition for, presence of, and/or risk of developing or suffering from endometriosis in a female subject. The present invention also relates to a kit useful for determining the risk of developing or suffering from endometriosis in a subject, a binding molecule specifically binding to the vitamin D-binding protein (DBP) GC*2 or DBP GC*1 allele product, and a binding molecule binding to the gene encoding the DBP GC*2 or GC*1 allele product.

Endometriosis, the ectopic presence of endometrial glands and stroma, is a common disease of reproductive age women. It affects about 10% of all women and upwards of 40% of women with infertility (Missmer, Obstet Gynecol Clin North Am (2003), 30: 1-19). The precise etiology of endometriosis remains unclear.

The most accepted theory regarding the pathophysiology of endometriosis is that it arises from retrograde menstruation, leading to the subsequent adhesion of endometrial tissues in the pelvis (Sampson, Am J Obstet Gynecol (1927), 14: 422-469). In recent years, this theory has been considerably extended and supplemented by studies demonstrating the significance of uterine autotraumatization of basal endometrium caused by hyperperistalsis activating basic tissue injury and repair mechanisns (TIAR) as well as by studies demonstrating that women with endometriosis may have an impairment of their immune systems and inflammatory responses, allowing for the development of endometriotic lesions (Seli, Semin Reprod Med (2003), 21: 135-144; Leyendecker, Hum Reprod (1996), 11: 1542-1551; Maeda, Fertil Steril (2002), 77: 679-683; Leyendecker, Arch Hynecol Obstet (2009), 280: 529-538).

The role of macrophages and their relation to endometriosis has been the subject of multiple studies (Akoum, Fertil Steril (2002), 77: 989-994; Rana, Fertil Steril (1996), 65: 925-930). McLaren et al. (McLaren, J Clin Invest (1996), 98: 482-489) showed that macrophages are highly expressed in the peritoneal fluid of women with endometriosis, stimulating angiogenesis and aiding in the implantation of endometriosis in the peritoneal cavity. Subsequent studies have confirmed macrophages' involvement in creating a positive loop for the establishment of endometriosis via the secretion of macrophage inflammatory protein MIP-1α, MIP-2, IL-6, TNF-α, and VEGF (Lin, Endocrinol (2006), 147: 1278-1286). A combination of markers that included macrophage chemotactic protein-1 (MCP-1) and macrophage migration inhibitory factor has been reported as a possible non-invasive test for endometriosis in some women (Seeber, Fertil Steril (2008), 89: 1073-1081).

In very recent years, a new approach to the study of endometriosis has been undertaken using genomics and proteomics, with the hope that differences between women with and without disease may shed light on novel pathways of disease, and possibly lead to the discovery of biomarkers. Families of genes associated with the immune system and inflammatory pathways, cell adhesion and extracellular matrix remodeling have been among those reported to be differentially expressed in endometriosis (Eyster, Fertil Steril (2007), 88: 1505-1533; Wren, Hum Reprod (2007), 22: 2093-2102). Proteomic analyses of peritoneal fluid have confirmed that inflammatory proteins, such as S100-A8, α1-antitrypsin, and apolipoprotein A-1, are elevated in women with endometriosis, but none of these proteins can be used as a biomarker for clinical diagnosis at this time (Ferrero, Gynecol Endocrinol (2008), 433-441; Ferrero, J Reprod Med (2009), 54: 32-40). Others have reported dysregulated gene and protein expression in eutopic and ectopic endometrium in women with endometriosis (Matsuzaki, Fertil Steril (2006), 86: 548-553; Fowler, Proteomics (2007), 7: 130-142), but again these were not a basis for a minimally invasive test of endometriosis (Sherwin, Hum Reprod (2008), 23: 1063-1068).

Few studies have applied proteomic techniques to the analysis of women with endometriosis (Zhang, Fertil Steril (2006), 86: 274-282; Liu, Fertil Steril (2007), 87: 988-990; Seeber, Fertil Steril (2010), 93: 2137-2144). Using high-through-put techniques, these studies have reported promising preliminary findings of differential protein expression in diseased women.

However, all have acknowledged the severe limitation of not being able to readily identify the proteins discovered. Thus, there is still a need for economic and feasible ways to diagnose and predict endometriosis in women.

### SUMMARY OF THE INVENTION

The present invention addresses and solves this problem by providing the means and methods as defined in the claims and as described and exemplified herein.

As described herein below, the present invention provides novel, simple and convenient means and methods for diagnosing and predicting endometriosis in a female subject.

These means and methods provided herein are based on the surprising findings of the present inventors who applied novel methods of protein expression analysis using two dimensional difference gel electrophoresis (2D-DIGE) to analyze serum of women with endometriosis. By allowing for multiple comparisons across gels, 2D-DIGE minimized gel to gel variation, increasing the accuracy and reproducibility of results. Utilizing spot analysis software which assigns statistical confidence to spot differences observed, the present inventors focused on protein spots of greatest interest for subsequent identification by mass spectrometry. Thereby, the present inventors identified novel biomarkers of endometriosis.

Particularly, in context with the present invention, 25 protein spots were found which exhibit a significant difference in abundance between women with endometriosis and controls, including acute phase proteins and complement components. As has been surprisingly found, the abundance of vitamin D-binding protein (DBP) was approximately 3-fold higher in all endometriosis pools compared to the control pool (p<0.02). Analysis of specific allele products using nanoLC-ESI-MS (nano liquid chromatography-electrospray-ionisation-mass spectrometry; see, e.g., Wuhrer, Int J Mass Spectrometry (2004), 232: 51-57) indicated that it was the GC*2 (“Group-specific Component 2”) allele product that was present in greater concentration in serum pools, as well as in single validation samples, in women with endometriosis (p=0.006). In contrast to the GC*1 (“Group-specific Component 1” [GC*1F and GC*1S]) allele products which are readily converted to a potent macrophage factor (Gc-MAF), the GC*2 allele product undergoes practically no such conversion (Yamamoto, J Immunol (1993), 151: 2794-2802). It can be speculated that the inability to sufficiently activate macrophages' phagocytotic function in those carrying the GC*2 allele (more prevalent in endometriosis) may allow endometriotic tissues to implant in the peritoneal cavity.

Accordingly, in context with the present invention, the GC*2 allele product was identified as a risk factor for endometriosis which can be used as a biomarker for diagnosing or predicting endometriosis in female subjects. Particularly, in context with the present invention, the GC*2 allele product can be used as a biomarker for determining the susceptibility to, predisposition for, presence of, and/or risk of developing or suffering from endometriosis in a female subject. Furthermore, as the skilled person is readily aware of, not only the GC*2 allele product, but also the GC*2 allele, i.e. the gene encoding the GC*2 allele product, can be used as a biomarker for the methods described herein. Also, transcription products of the GC*2 allele can be used as biomarkers for the methods described and provided herein. For example, a transcription product of the GC*2 allele may be an mRNA as transcribed from the GC*2 allele. The same applies mutatis mutandis for fragments of the GC*2 allele product, the GC*2 allele and transcription products of the GC*2 allele which can also be used as biomarkers in context with the means and methods described and provided herein. The terms “GC*2 allele product”, “GC*2 allele” and “transcription product” as used herein are to be construed as commonly used by the person skilled in the art and as further defined herein below unless indicated otherwise. In case of discrepancy, the terms “GC*2 allele product”, “GC*2 allele” and “transcription product” shall be construed as described herein.

The present invention therefore provides a method for determining the susceptibility to, predisposition for, presence of, and/or risk of developing or suffering from endometriosis in a female subject, said method comprising the steps of
(a) obtaining a biological sample from said subject; and
(b) determining the presence of
   (i) the vitamin D-binding protein (DBP) GC*2 allele product or a fragment thereof;
   (ii) the DBP GC*2 allele or a fragment thereof; or
   (iii) a transcription product, preferably an mRNA, of the DBP GC*2 allele, or a fragment of said transcription product
   in said sample,
      wherein the presence of the DBP GC*2 allele gene product or fragment thereof, the DBP GC*2 allele or fragment thereof, or transcription product of the DBP GC*2 allele or fragment thereof is indicative for an increased susceptibility to, predisposition for and/or risk of developing or suffering from endometriosis and/or for the presence of endometriosis.

The present invention also provides a method for diagnosing endometriosis in a female subject, said method comprising the steps of
(a) obtaining a biological sample from said subject; and
(b) determining the presence of
   (i) the vitamin D-binding protein (DBP) GC*2 allele product or a fragment thereof;
   (ii) the DBP GC*2 allele or a fragment thereof; or
   (iii) a transcription product, preferably an mRNA, of the DBP GC*2 allele, or a fragment of said transcription product
   in said sample,
      wherein the presence of the DBP GC*2 allele gene product or fragment thereof, the DBP GC*2 allele or fragment thereof, or transcription product of the DBP GC*2 allele or fragment thereof is indicative for an increased susceptibility to, predisposition for and/or risk of developing or suffering from endometriosis and/or for the presence of endometriosis.

The present invention also provides a method for predicting endometriosis in a female subject, said method comprising the steps of
(a) obtaining a biological sample from said subject; and
(b) determining the presence of
   (i) the vitamin D-binding protein (DBP) GC*2 allele product or a fragment thereof;
   (ii) the DBP GC*2 allele or a fragment thereof; or
   (iii) a transcription product, preferably an mRNA, of the DBP GC*2 allele, or a fragment of said transcription product
   in said sample,
      wherein the presence of the DBP GC*2 allele gene product or fragment thereof, the DBP GC*2 allele or fragment thereof, or transcription product of the DBP GC*2 allele or fragment thereof is indicative for an increased susceptibility to, predisposition for and/or risk of developing or suffering from endometriosis and/or for the presence of endometriosis.

As has been found in context with the present invention and as also described herein, donors showing only the GC*2 allele product and no GC*1 allele product (homozygous for the GC*2 allele) were only found in the endometriosis groups and not in the control groups. Accordingly, homozygous GC*2 allele carrying female subjects appear to have an even highly increased susceptibility to, predisposition for, presence of, and/or risk of developing or suffering from endometriosis. Furthermore, it context of the present invention it was found that total DBP product produced appears to be stable (Borkowski, Postepy Hig Med Dosw (2008), 62: 103-109); see also FIG. 6. Accordingly, the skilled person, by comparing the determined amount of GC*2 allele product with the amount of total DBP to be expected (or from a control donor), can readily determine whether the analyzed sample is from a donor carrying one copy of the GC*2 allele (heterozygous donor) or two copies of the GC*2 allele (homozygous donor). Thus, the abundance of GC*2 allele product may indirectly indicate whether GC*1 product is also being expressed (heterozygous condition) since the total DBP product produced appears to be stable.

As an alternative method to determine whether a female subject to be analyzed is homozygous or heterozygous for the GC*2 allele, the presence of the GC*1F and/or GC*1S allele product or fragment thereof, the GC*1F or GC*1S allele or fragment thereof, or transcription product (e.g., mRNA) of the GC*1F or GC*1s allele or fragment thereof (i.e. fragment of said transcription product) may be determined in addition to the corresponding GC*2 pendants.

Accordingly, the method described herein may further comprise the step of
(c) determining the presence of
   (i) the vitamin D-binding protein (DBP) GC*1F or GC*1S allele product or a fragment thereof;
   (ii) the DBP GC*1F or GC*1S allele or a fragment thereof; or
   (iii) a transcription product, preferably an mRNA, of the DBP GC*1F or GC*1S allele, or a fragment of said transcription product
   in said sample,
      wherein the presence of the DBP GC*2 allele product or fragment thereof, the DBP GC*2 allele or fragment thereof, or transcription product of the GC*2 allele or fragment thereof,
      and the absence of DBP GC*1F or GC*1S allele product or fragment thereof, or DBP GC*1F or GC*1S allele or fragment thereof, or transcription product of the DBP GC*1F or GC*1S allele or fragment thereof is indicative for a highly increased susceptibility to, predisposition for and/or risk of developing or suffering from endometriosis and/or for the presence of endometriosis.

As further described herein, in context with the present invention, the means and methods provided herein may be applied for diagnosing female subjects such as humans and other mammals. In a preferred embodiment of the present invention, the female subject is human. In a less preferred embodiment of the present invention, the subject is a male, preferably a human male, in particular a male which has been treated with estrogen.

Generally, in context with the present invention, there are several methods well known in the art how to carry out the respective steps of the methods described and provided herein. For example, determining the presence of the DBP GC*2, GC*1F or GC*1S allele product or a fragment thereof may be performed by methods such as RIA (Radio Immuno Assay), sandwich (immunometric assay), Western blot, IRMA (Immune Radioimmunometric Assay), EIA (Enzyme Immuno Assay), ELISA (Enzyme Linked Immuno Assay), FIA (Fluorescent Immuno Assay), CLIA (Chemioluminescent Immune Assay), 2D-Gel, mass spectrometry, including MALDI/TOF, or the like. In one embodiment, the method of choice in this context is an ELISA. These methods may also be used to determine the presence of the DBP GC*2, GC*1F or GC*1S allele or a fragment thereof as well as the presence of a transcription product such as an mRNA of the DBP GC*2, GC*1F or GC*1S allele or a fragment thereof. Binding molecules and kits containing binding molecules suitable to perform these methods are further described and exemplified herein.

Furthermore, in context with the present invention, determining the presence of the DBP GC*2, GC*1F or GC*1S allele or a fragment thereof, or transcription products of the DBP GC*2, GC*1F or GC*1S allele or a fragment thereof (i.e. of the transcription product) may be performed by methods such as PCR, qPCR, RT-PCR, qRT-PCR, RT-qPCR, sequencing (optionally subsequent to a PCR qPCR, RT-PCR, qRT-PCR or RT-qPCR), Light Cycler®, TaqMan® Platform and Assays or quantigene assay (Zhou, Anal Biochem (2000), 282: 46-53), FISH, Northern blot, dot blot, microarrays, next generation sequencing (VanGuilder, Biotechniques (2008), 44(5): 619-26; Elvidge, Pharmacogenomics (2006), 7: 123-134; Metzker, Nat Rev Genet (2010), 11: 31-46; Kafatos, NAR (1979), 7: 1541-1552), or the like. In one embodiment, the method of choice in this context is a PCR.

As further described herein, in context with the present invention, the biological sample to be analyzed may be selected from the group consisting of blood, serum, plasma, blood cells, other blood derived products, saliva, vaginal fluid, urine, cerebrospinal fluid, and the like. For example, if the presence of the GC*1F, GC*1S or GC*2 allele product or fragment thereof is to be determined, the biological sample is in one embodiment serum. If the presence of GC*1F, GC*1S or GC*2 allele or fragment thereof, or transcription product such as mRNA of the GC*1F, GC*1S or GC*2 allele is to be determined, the biological sample is in one embodiment blood cells.

As further described herein, in context with the present invention, particularly the GC*1 allele products may be glycosylated. The site of glycosylation may be, e.g., the threonine at position of 418 of SEQ ID NOs. 3 and 4 (GC*1F or GC*1S, respectively) and/or, particularly, the threonine (Thr, T) at position of 420 of SEQ ID NOs. 3 and 4 (GC*1F or GC*1S, respectively). The threonine at position 418 may be glycosylated with, e.g., a GalNAc/Gal disaccharide. The threonine at position 420 may be glycosylated with, e.g., GalNAc/Gal disaccharide or GalNAc/Gal/NeuNAc trisaccharide.

The present invention further provides for a kit useful for determining the risk of developing or suffering from endometriosis and/or the presence of endometriosis in a subject, said kit comprising one or more binding molecules specifically binding to the GC*2 allele product or a fragment thereof, the GC*2 allele or a fragment thereof, and/or a transcription product of GC*2 allele or a fragment thereof.

Accordingly, the present invention also relates to a binding molecule specifically binding to the DBP GC*2 allele product, or the GC*1F and/or GC*1S allele product. Preferably, a binding molecule in context with the present invention is capable to differentiate between GC*1F and/or GC*1S allele product on the one hand and GC*2 allele product on the other hand. For example the binding molecule of the present invention specifically binds to GC*1F allele product and/or GC*1S allele product but not to GC*2 allele product. In another example, the binding molecule of the present invention specifically binds to the GC*2 allele product but neither to GC*1F allele product nor GC*1S allele product. Also, in context with the present invention, the same applies for respective fragments of the GC*1 or GC*2 allele products, as long as the binding molecule is able to differentiate between a fragment of GC*2 allele product on the one hand and GC*1F and/or GC*1S allele products on the other hand. In this context, a fragment of GC*1 or GC*2 allele products preferably comprises a fragment of SEQ ID NO: 1, SEQ ID NO: 3 or SEQ ID NO: 4, respectively, containing the amino acid at position 420. Such fragments may be at least 5 amino acids, preferably at least 10, 15, 20, 25, 40, 50, 60, 70, 80, 90, 100, 125, 150, 175 or 200 amino acids in length. Fragments of GC*1 and GC*2 allele products are further described and exemplified herein.

Binding molecules in context of the present invention may be antibodies as further described herein.

The present invention further provides for binding molecule specifically binding to the gene encoding the DBP GC*2 allele or fragments thereof, or the GC*1F and/or GC*1S allele or fragments thereof as well as to transcription products of these alleles or fragments thereof (i.e. of the transcription product). As further described herein, such binding molecules may be molecules such as primers or probes. In context with the present invention, such primers may be useful in the methods described and provided herein, e.g., for performing PCR. Probes may be useful in the methods described and provided herein, e.g., for performing microarray.

### BRIEF DESCRIPTION OF THE FIGURES

FIG. 1: 2D-Gel image of serum depleted with Agilent's Hu-14. The marked spots differ statistically significantly by a factor of at least 2 between one of the endometriosis pools and the control pool. Spots 1 to 5, 7 to 10, 12, 18 and 22 were highest in the severe (stage IV) and moderate disease stage (stage III). Spots 6, 11, 13 to 17, 19 to 21 and 23 to 25 were highest in the control pool.

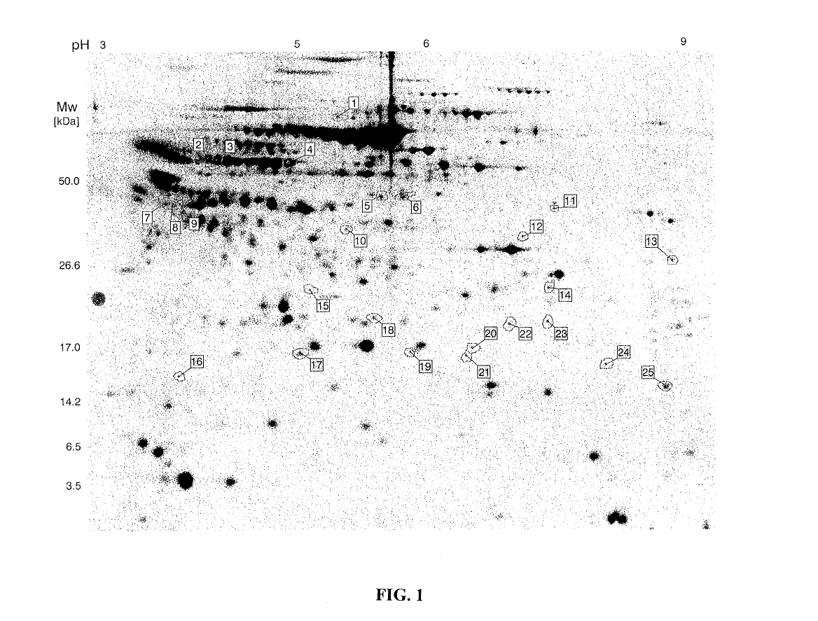

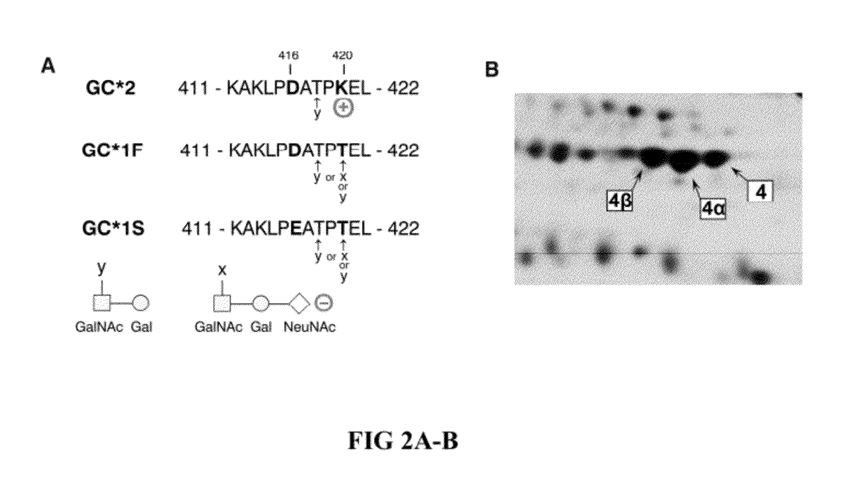

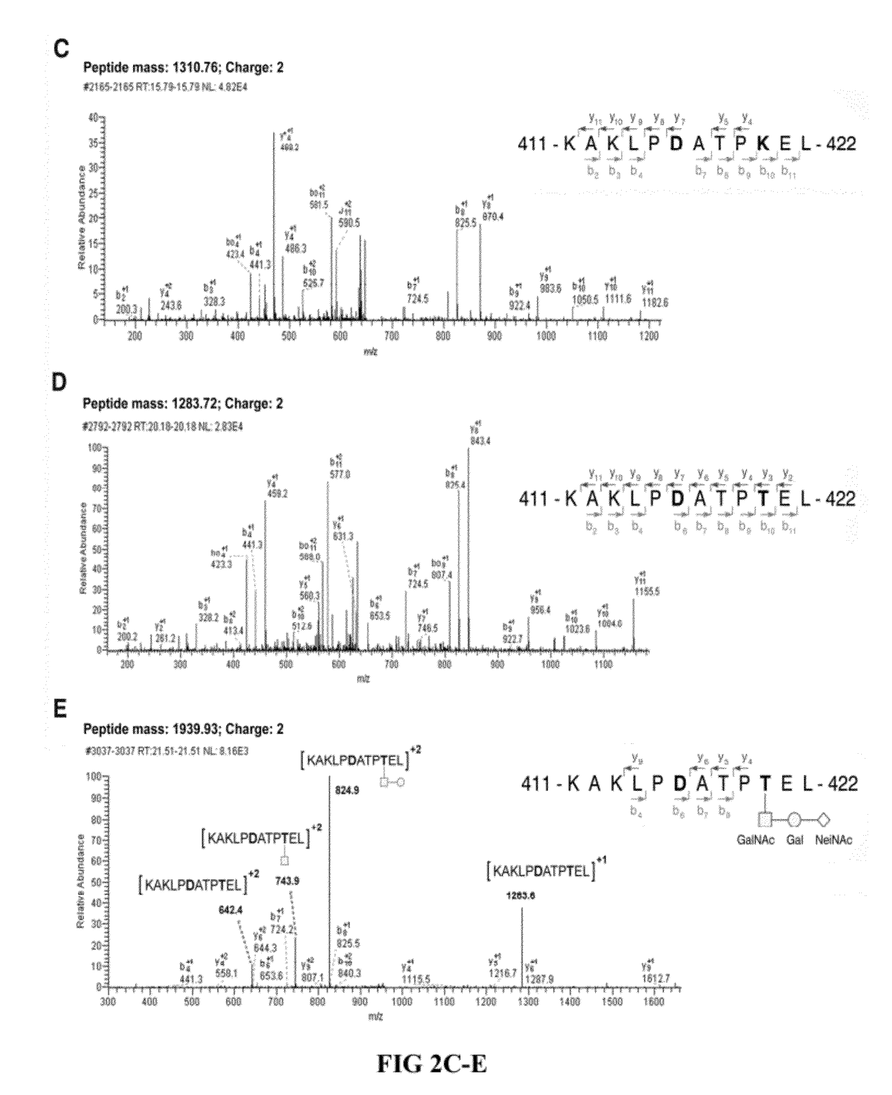

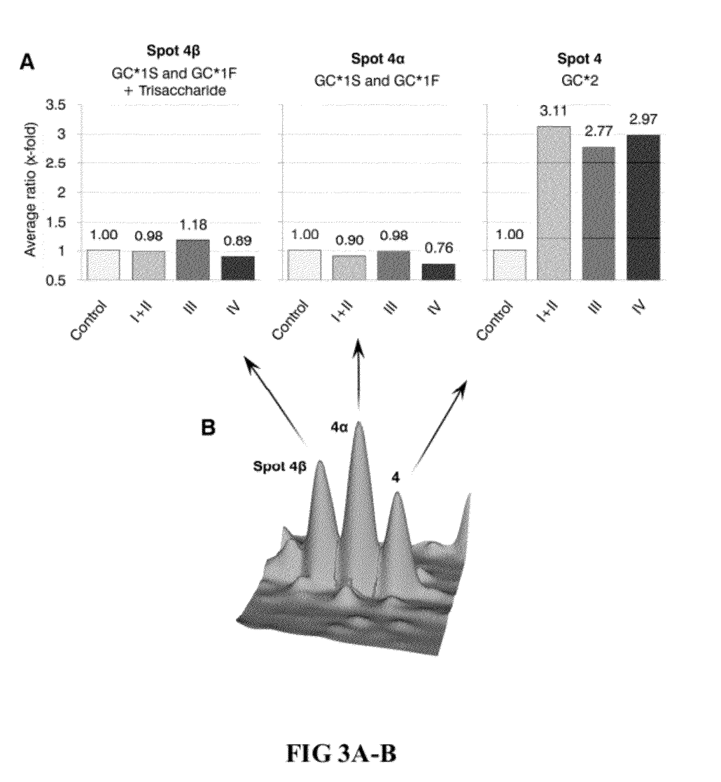

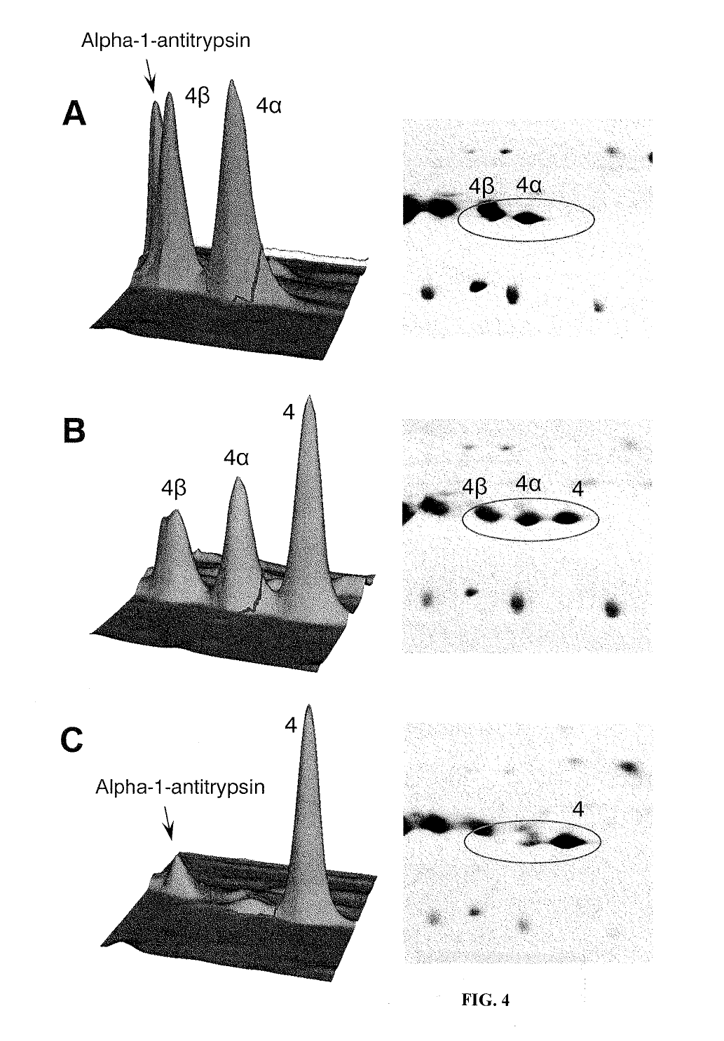

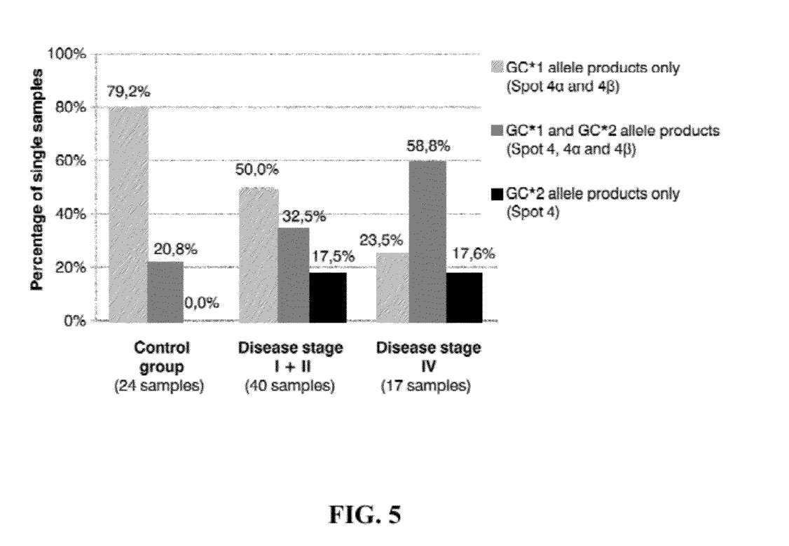

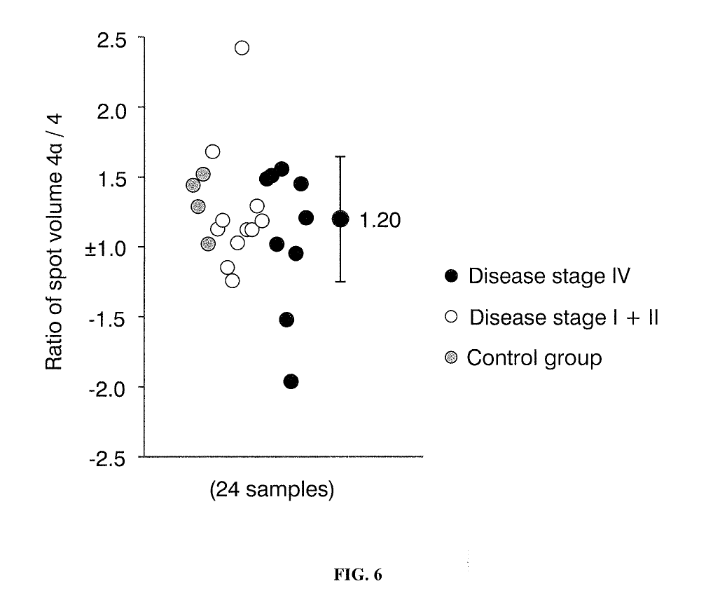

FIG. 2: Mass spectrometry identification of the three major DBP allele products (GC*1F, GC*1S, GC*2).
(A) Known primary structures of the three major DBP allele products differing at two positions, 416 and 420. Arrows donate sites of possible modification with (x) tri- or (y) disaccharide.
(B) Detail of FIG. 1. Spot 4 contained the DBP allele product GC*2, spot 4α contained GC*1F and GC*1S unmodified and spot 4β contained GC*1F and GC*1S modified with the linear trisaccharide.
(C) MS/MS spectrum of the GC*2 specific amino acid sequence identified in spot 4.
(D) MS/MS spectrum of the GC*1F specific amino acid sequence identified in spot 4α and
(E) MS/MS spectrum of the GC*1F specific amino acid modified with the linear trisaccharide identified in spot 4β.

FIG. 3: (A) Average ratio of DBP allele products in the endometriosis pools relative to the control pool in the three spots belonging to the DBP chain. Corresponding p-values for between group differences of spot 4 can be seen in Table 1.
(B) 3D rendered detail of the three DBP spots illustrating the spot volume and the quality of separation using a pH range from 3 to 11 for isoelectric focusing (IEF).

FIG. 4: Appearance of DBP spots in single samples. Three combinations occurred:
(A) Only GC*1 allele products (spot 4α and 4β);
(B) both allele products (all three spots); or
(C) only the GC*2 allele product (spot 4).

FIG. 5: Frequency of the allelic forms observed in the depleted single samples allocated to the disease stage. The distribution was significantly different between the groups evaluated (stage VII, stage IV, and control), p=0.00.

FIG. 6: Relative ratio of the spot volume 4 to spot 4α of single samples expressing both allele products, as seen in FIG. 4B. A positive value signifies that the volume of spot 4 was greater than 4α. A negative value means that the spot volume of 4α was greater.

### DETAILED DESCRIPTION OF THE INVENTION

Up to now, there have been only two studies that have evaluated DBP in endometriosis. Borkowski et al. (Borkowski, Postepy Hig Med Dosw (2008), 62: 103-109) compared total concentrations of DBP in serum and peritoneal fluid of women with and without endometriosis and found no differences. Furthermore, this study was unable to differentiate between different allele products of DBP. The other study of DBP used 2DE and reported that one isoform of DBP was significantly lower in the peritoneal fluid, but not in plasma, of women with endometriosis compared to controls (Ferrero, J Soc Gynecol Investig (2005), 12: 272-277). However, this study of Ferrero et al. did not identify which of the allele products of DBP was differentially expressed, relying only on the relative position of the protein spot on the gel.

In the present invention, a number of proteins was found that differed in abundance in serum between women with and without endometriosis. Surprisingly, it was found that there is a differential abundance of a specific allele product of vitamin D-binding protein (DBP), namely the GC*2 allele product. As described and shown herein, the expression of the GC*2 allele product was approximately 3-fold higher in all endometriosis pools compared to the control pool. Single sample analysis showed that in the control group, no subject expressed only the GC*2 allele product and approximately 20% of control women (without endometriosis) expressed both, GC*1 and GC*2 allele products, implying that the latter were heterozygous carriers of the GC*2 polymorphism, i.e. they carry both, one GC*1 allele and one GC*2 allele in their genome. This is consistent with the reported carrier rate in the population (White, Trends Endocrinol Metab (2000), 11: 320-327). In contrast, in the endometriosis group, nearly 18% of analyzed women expressed only the GC*2 allele product, and 32.5% and 58.8% expressed GC*2 allele product in combination with GC*1 allele products (heterozygous carriers), in stage I/II and stage IV disease, respectively.

Besides acting as a transporter for vitamin-D metabolites, DBP is the most potent activator for macrophages in the form of GcMAF (Gc Protein-derived Macrophage Activating Factor) (Arnaud, Hum Genet (1993), 92: 183-188; Yamamoto, Cell Immunol (1996), 170: 161-167). The three major allele products of DBP (GC*1S, GC*1F and GC*2) differ in their primary structure at two positions, 416 and 420 and, consequently, in their glycosylation state. In both, GC*2 and GC*1F allele product, there is a aspartic acid (Asp, D) at position 416, while in the GC*1S allele product, there is a glutamic acid (Glu, E) at position 416. In the GC*2 allele product, there is a (non-glycosylated) lysine (Lys, K) at position 420. In GC*1F and GC*1S allele products, there is a threonine (Thr, T) at position 420, which is often glycosylated. The threonine (Thr, T) at position 420 in the GC*1 allele products is mostly glycosylated by a GalNAc/Gal disaccharide or a GalNAc/Gal/NeuNAc trisaccharide, preferably O-linked. The macrophage activating function depends on the glycolysation state, i.e. most likely determined by a single GalNAc resulting when DBP is deglycosylated by T- and B-cell glycosidases (Berkkanoglu, Am J Reprod Immunol (2003), 50: 48-59). The GC*1 allele products are glycosylated at a total rate of 10-30% (altogether at T420 and T418), while the GC*2 allele product at a rate of 1-5% (at the T418) (Borges, J Proteome Res (2008), 7: 4143-4153). Thus, the form of DBP encoded by the GC*1 allele is much more readily converted to GcMAF, while that encoded by GC*2 is nearly not converted at all.

As described herein, in context with the present invention it was surprisingly found that a much higher percentage of women with endometriosis expressed the GC*2 allele product, both as the only protein expressed (in approximately 17.5%, independent of stage) or in combination with the GC*1 allele products (in upwards of 59%), while none of the women in the control group expressed only GC*2 allele products. It follows that those expressing only GC*2 allele products, disproportionally represented in the endometriosis group, have a much reduced capability to convert DBP to GcMAF, the critical macrophage activator.

Accordingly, as mentioned above, in context with the present invention, the GC*2 allele product was identified as a risk factor for endometriosis which can be used as a biomarker for diagnosing or predicting endometriosis in female subjects. Particularly, in context with the present invention, the GC*2 allele product can be used as a biomarker for determining the susceptibility to, predisposition for, presence of and/or risk of developing or suffering from endometriosis in a female subject. This will be further detailed and described herein below.

Unless indicated otherwise, the terms and keywords used herein shall generally be construed in their broadest meaning they usually have in the art. For further clarification, in the following, non-limiting definitions of terms and keywords used herein are provided.

The term “polynucleotide” or “nucleic acid molecule” as used herein includes DNA molecules, RNA molecules, or other nucleic acid molecules that comprise a stretch of nucleic acid residues, for example, nucleic acid molecules with modified backbones such as PNA molecules, GNA molecules, TNA molecules, LNA molecules, morpholino nucleic acid molecules, polysiloxane, and 2′-O-(2-methoxy)ethyl-phosphorothioate, comprising non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection. As used herein, polynucleotides may be in single- or double stranded form, in sense or antisense orientation. Deviations from the above-described nucleic acid sequences may have been produced, e.g., by deletion, substitution, addition, insertion of nucleotides and/or by recombination. The term “addition” refers to adding at least one nucleic acid residue to one or both ends of the given sequence, whereas “insertion” refers to inserting at least one nucleic acid residue within a given nucleotide sequence. The term “deletion” refers to deleting or removal of at least one nucleic acid residue in a given nucleotide sequence. The term “substitution” refers to the replacement of at least one nucleic acid residue in a given nucleotide sequence.

The term “nucleic acid residue” as used herein includes naturally occurring nucleic acid residues or artificially produced nucleic acid residues. Generally, examples for nucleic acid residues are adenine (A), guanine (G), cytosine (C), thymine (T), uracil (U), xanthine (X), and hypoxanthine (HX). In context of the present invention, thymine (T) and uracil (U) may be used interchangeably depending on the respective type of polynucleotide. For example, as the skilled person is aware of, a thymine (T) as part of a DNA corresponds to an uracil (U) as part of the corresponding transcribed mRNA.

The term “fragment of a polynucleotide” as used herein includes a nucleic acid molecule that comprises a stretch of contiguous nucleic acid residues comprised by the complete polynucleotide referred to. For example, “fragment of a polynucleotide” may refer to a nucleic acid molecule comprising a nucleic acid sequence of at least 5 nucleic acid residues, preferably at least 10, 15, 20, 25, 40, 50, 60, 70, 80, 90, 100, 125, 150, 175, 200, 250, 300, 350, 400, 450, 500, 550, 600, 650, 700, or 750 nucleic acid residues of the nucleic acid sequence of said complete polynucleotide.

The term “polypeptide” or “amino acid sequence” as used herein includes proteins, proteinaceous molecules, fragments of proteins, monomers, subunits or portions of polymeric proteins, peptides, oligopeptides, or any other molecule comprising a stretch of amino acid residues linked by peptidic bonds. Deviations from amino acid sequences may have been produced, e.g., by deletion, substitution, addition, insertion of amino acids. The term “addition” refers to adding at least one amino acid residue to one or both ends of the given sequence, whereas “insertion” refers to inserting at least one amino acid residue within a given amino acid sequence. The term “deletion” refers to deleting or removal of at least one amino acid residue in a given amino acid sequence. The term “substitution” refers to the replacement of at least one amino acid residue in a given amino acid sequence.

The term “amino acid residue” as used herein includes any amino acid which can be part of an amino acid sequence and includes naturally occurring amino acids and artificial amino acids. Generally, amino acid residues comprise an amine group, a carboxylic acid group and a side chain that may vary between different amino acids residues. The amino acids may be proteinogenic, i.e. part of naturally occurring polypeptides or proteins. Non-limiting examples for naturally occurring amino acids are alanine (Ala, A), arginine (Arg, R), aspartic acid (Asp, D), asparagine (Asn, N), cysteine (Cys, C), glutamic acid (Glu, E), glutamine (Gln, Q), glycine, (Gly, G), histidine (His, H), isoleucine (Iso, I), leucine (Leu, L), lysine (Lys, K), methionine (Met, M), phenylalanine (Phe, F), proline (Pro, P), serine (Ser, S), threonine (Thr, T), tryptophane (Trp, W), tyrosine (Tyr, Y), valine (Val, V), selenocysteine (Sec, U), pyrrolysine (Pyl, O), ornithine, taurine, and citrulline.

The term “fragment of a polypeptide” or “fragment of an amino acid sequence” as used herein includes a peptide, an oligopeptide or a polypeptide that comprises a stretch of contiguous amino acid residues comprised by the complete polypeptide referred to. For example, “fragment of a polypeptide” may refer to a peptide, an oligopeptide or a polypeptide comprising an amino acid sequence of at least 5 amino acid residues, preferably at least 10, 15, 20, 25, 40, 50, 60, 70, 80, 90, 100, 125, 150, 175, 200, 250 amino acid residues of the amino acid sequence of said complete polypeptide.

The term “transcription product” as used herein includes nucleic acid molecules which are transcribed from a nucleotide sequence, allele or gene. For example, a transcription product referred to herein may be an mRNA. An mRNA as used herein is not limited to particular splice variants but is preferably in a form which can be translated into a polypeptide.

The meanings of the terms “(DBP) GC*2 allele”, “(DBP) GC*1 allele”, “(DBP) GC*1F allele” and “(DBP) GC*1S allele” are known in the art. These terms are to be understood accordingly in the context of the present invention. In particular, these terms are to be understood in the context of the present invention as follows:

“(DBP) GC*2 allele” or the transcription product thereof comprises a nucleic acid molecule selected from the group consisting of
(a) a nucleic acid molecule comprising SEQ ID NO: 2;
(b) a nucleic acid molecule encoding an amino acid sequence comprising SEQ ID NO: 1; and
(c) a nucleic acid molecule hybridizing under (highly) stringent conditions to a nucleic acid molecule of any one of (a) and (b).

“DBP GC*2 allele product” comprises an amino acid sequence selected from the group consisting of
(a) an amino acid sequence comprising SEQ ID NO: 1;
(b) an amino acid sequence encoded by a nucleic acid molecule of SEQ ID NO: 2; and
(c) an amino acid sequence encoded by a nucleic acid molecule hybridizing under (highly) stringent conditions to a nucleic acid molecule of SEQ ID NO: 2 or to a nucleic acid molecule encoding an amino acid sequence as defined in (a) or (b).

The “DBP GC*1S allele” or the transcription product thereof comprises selected from the group consisting of a nucleic acid molecule
(a) a nucleic acid molecule comprising SEQ ID NO: 6;
(b) a nucleic acid molecule encoding an amino acid sequence comprising SEQ ID NO: 3; and
(c) a nucleic acid molecule hybridizing under (highly) stringent conditions to a nucleic acid molecule of any one of (a) and (b).

The “DBP GC*1S allele product” comprises an amino acid sequence selected from the group consisting of
(a) an amino acid sequence comprising SEQ ID NO: 3;
(b) an amino acid sequence encoded by a nucleic acid molecule of SEQ ID NO: 6; and
(c) an amino acid sequence encoded by a nucleic acid molecule hybridizing under (highly) stringent conditions to a nucleic acid molecule of SEQ ID NO: 6 or to a nucleic acid molecule encoding an amino acid sequence as defined in (a) or (b).

The “DBP GC*1F allele” or the transcription product thereof comprises selected from the group consisting of a nucleic acid molecule
(a) a nucleic acid molecule comprising SEQ ID NO: 5;
(b) a nucleic acid molecule encoding an amino acid sequence comprising SEQ ID NO: 4; and
(c) a nucleic acid molecule hybridizing under (highly) stringent conditions to a nucleic acid molecule of any one of (a) and (b).

The “DBP GC*1F allele product” comprises an amino acid sequence selected from the group consisting of
(a) an amino acid sequence comprising SEQ ID NO: 4;
(b) an amino acid sequence encoded by a nucleic acid molecule of SEQ ID NO: 5; and
(c) an amino acid sequence encoded by a nucleic acid molecule hybridizing under (highly) stringent conditions to a nucleic acid molecule of SEQ ID NO: 5 or to a nucleic acid molecule encoding an amino acid sequence as defined in (a) or (b).

The term “fragment of a transcription product” or “fragment of an mRNA” as used herein, be it in context with the transcription product or mRNA of the GC*1F, GC*1S or GC*2 allele, includes a polynucleotide comprising a stretch of consecutive nucleic acids comprised in the respective full-length transcription product or mRNA of the respective GC allele. Generally, when using terms like “transcription product of an allele (e.g., GC*1F, GC*11S or GC*2 allele) or fragment thereof”, the term ‘fragment thereof’ refers to a fragment of the transcription product, not to a fragment of the respective allele. In this context, a fragment of a transcription product or mRNA may comprise or consist of at least 5 nucleic acids, preferably at least 10, 15, 20, 25, 40, 50, 60, 70, 80, 90, 100, 125, 150, 175, 200, 250, 300, 350, 400, 450, 500, 550, 600, 650, 700 nucleic acids. In context of the present invention, a fragment of a transcription product or mRNA preferably comprises the nucleic acid which corresponds to the nucleic acid at position 1565 (A in GC*2/SEQ ID NO: 2; C in GC*1F/SEQ ID NO: 5 and GC*1S/SEQ ID NO: 6) of the corresponding GC allele, i.e. SEQ ID NO: 2 (GC*2 allele), SEQ ID NO: 5 (GC*1F allele) or SEQ ID NO: 6 (GC*1S allele). Additionally, the fragment of a transcription product or mRNA may comprise the nucleic acid which corresponds to the nucleic acid at position 1554 (T in GC*2/SEQ ID NO: 2 and GC*1F/SEQ ID NO: 5; G in GC*1S/SEQ ID NO: 6).

The term “gene” as used herein includes a nucleotide sequence which comprises a stretch of nucleic acid residues including a start codon and a stop codon. Preferably, in context with the present invention, a gene encodes an amino acid sequence of a corresponding polypeptide or protein. Preferably, the transcription product of a gene is an mRNA. An mRNA as used herein is not limited to particular splice variants but is preferably in a form which can be translated into a polypeptide or protein. Generally, a gene may exist in the form of different alleles.

The term “allele” as used herein includes a particular nucleotide sequence of a gene which exists in two or more forms. For example, as it is known in the art, there are different alleles for the vitamin D-binding protein (DBP) gene (Group Specific Component (GC)), i.e. there is a genetic polymorphism (Cleve, Vox Sang (1988), 54: 215-225). Particularly, for the gene encoding DBP, there exist at least three alleles, namely GC*1F (SEQ ID NO: 5), GC*1S (SEQ ID NO: 6), and GC*2 (SEQ ID NO: 2). In context with the present invention, when referred to “GC*1 allele”, “GC*1F allele”, “GC*1S allele” or “GC*2 allele”, reference is made to a respective allele of the vitamin D-binding protein (DBP) gene.

The term “fragment of a GC allele”, as used herein, be it in context with GC*1, GC*1F, GC*1S or GC*2, includes a polynucleotide comprising a stretch of consecutive nucleic acids comprised in the respective full-length GC allele (GC*1F: SEQ ID NO: 5; GC*1S: SEQ ID NO: 6 or GC*2: SEQ ID NO: 2, respectively). In this context, a fragment of a GC allele may comprise or consist of at least 5 nucleic acids, preferably at least 10, 15, 20, 25, 40, 50, 60, 70, 80, 90, 100, 125, 150, 175, 200, 250, 300, 350, 400, 450, 500, 550, 600, 650, 700 nucleic acids. In context of the present invention, a fragment of a GC allele preferably comprises the nucleic acid at position 1565 of D NO: 2, SEQ ID NO: 5 or SEQ ID NO: 6, respectively. The fragment of a GC allele may additionally comprise the nucleic acid at position 1554 of D NO: 2, SEQ ID NO: 5 or SEQ ID NO: 6, respectively.

The term “allele product” as used herein includes a polypeptide which is encoded by a particular allele. For example, the allele product of the GC*1F allele (also referred to herein as “GC*1F allele product”) is a polypeptide encoded by the GC*1F allele (SEQ ID NO: 5) and has an amino acid sequence as shown in SEQ ID NO: 3. The allele product of the GC*1S allele (also referred to herein as “GC*1S allele product”) is a polypeptide encoded by the GC*1S allele (SEQ ID NO: 6) and has an amino acid sequence as shown in SEQ ID NO: 4. As another example, the allele product of the GC*2 allele (also referred to herein as “GC*2 allele product”) is a polypeptide encoded by the GC*1 allele (SEQ ID NO: 2) and has an amino acid sequence as shown in SEQ ID NO: 1. In context with the present invention, when referred to “GC*1 allele product”, “GC*1F allele product”, “GC*1S allele product” or “GC*2 allele product”, reference is made to a respective allele product of the vitamin D-binding protein (DBP).

The term “fragment of a GC allele product”, as used herein, be it in context with GC*1, GC*1F, GC*1S or GC*2, includes a polypeptide comprising a stretch of consecutive amino acids comprised in the respective full-length GC allele product (GC*1F: SEQ ID NO: 3; GC*1S: SEQ ID NO: 4 or GC*2: SEQ ID NO: 1, respectively). In this context, a fragment of a GC product allele may comprise or consist of at least 5 amino acids, preferably at least 10, 15, 20, 25, 40, 50, 60, 70, 80, 90, 100, 125, 150, 175 or 200 amino acids. In context of the present invention, a fragment of a GC product allele preferably comprises the amino acid at position 420 of SEQ ID NO: 1, SEQ ID NO: 3 or SEQ ID NO: 4, respectively. As an example, the fragment of a GC allele product may comprise or consist of amino acids 411 to 422 of SEQ ID NO: 1 (for GC*2 allele product fragment), SEQ ID NO: 3 (for GC*1F allele product fragment) or SEQ ID NO: 4 (for GC*15 allele product fragment).

The term “subject” as used herein relates to female humans and other female mammals. In the context of this invention, it is particularly envisaged that such mammals are economically, agronomically or scientifically important. Scientifically important organisms include, but are not limited to, mice, rats, and rabbits. Non-limiting examples of agronomically important animals are sheep, cattle and pigs. Cats and dogs may exemplarily be considered as economically important animals. Preferably, in context with the present invention, the subject is a female human. In a less preferred embodiment of the present invention, the subject is a male, preferably a human male, in particular a male which has been treated with estrogen.

The term “biological sample” as used herein includes blood, serum, plasma, other blood derived products, saliva, vaginal fluid, urine, cerebrospinal fluid, peritoneal fluid or the like. Preferably, the sample is blood. Most preferably, the sample is serum for immuno assays (e.g., ELISA) or blood cells for genetic assays (e.g., PCR).

The term “binding molecule” as used herein includes, but is not limited to, antibodies and nucleic acid molecules. Binding molecules in context with the present invention may specifically bind to GC*2 allele product or fragment thereof, GC*2 allele or fragment thereof, or transcription product (e.g., mRNA) of GC*2 allele or fragment thereof (i.e. of the transcription product). Furthermore, in context of GC*1F and/or GC*1S allele, binding molecules may specifically bind to GC*1F and/or GC*1S allele product or fragment thereof, GC*1F and/or GC*1S allele or fragment thereof, or transcription product (e.g., mRNA) of GC*2 allele or fragment thereof (i.e. of the transcription product). In context with the present invention, a binding molecule is able to distinguish between GC*1F (allele, allele product, transcription product of GC*1F allele, or corresponding fragments thereof) and/or GC*1S (allele, allele product, transcription product of GC*1S allele, or corresponding fragments thereof) on the one hand and GC*2 (allele, allele product, transcription product of GC*2 allele, or corresponding fragments thereof) on the other hand. Preferably, in context with the present invention, nucleic acid molecules as “binding molecules” are able to hybridize under non-stringent or, preferably, stringent conditions to a GC allele (GC*2: SEQ ID NO: 2, GC*1F: SEQ ID NO: 5 or GC*1S: SEQ ID NO: 6) or a fragment thereof and/or is complementary thereto. A nucleic acid molecule as a “binding molecule” may comprise at least 5 consecutive nucleic acid residues, preferably at least 10, 15, 20, 25, 40, 50, 60, 70, 80, 90, 100, 125, 150, 175, 200, 250, 300, 350, 400, 450, 500, 550, 600, 650, 700 nucleic acid residues. In one embodiment, a nucleic acid molecule as a “binding molecule” is a primer binding to a GC allele (GC*2: SEQ ID NO: 2, GC*1F: SEQ ID NO: 5 or GC*1S: SEQ ID NO: 6) or a fragment thereof. In this context, a primer is preferably 20 to 30 nucleotides in length. Examples for primers suitable for performing the method described and provided herein are 59-TGTAGTAAGACCTTACATTTAAATGG-39 (forward) (SEQ ID NO: 7), and 59-TACGTTCTTAAAAGATTCTGCCATG-39 (reverse) (SEQ ID NO: 8); see Baier, J Clin Endocrinol Metab (1998), 83: 2993-2996.

As further described herein, such a primer may be used, e.g., in PCR assays or the like to determine the presence of a GC*1F, GC*1S, or GC*2 allele or a transcription product (e.g., mRNA) thereof. Genotyping of DBP using PCR is well known in the art and has also been described by previous studies; see, e.g., Ito, Chest (2004), 125: 63-70 or Pani, JCEM (2002), 87: 2564-2567. In another embodiment, the nucleic acid molecule as “binding molecule” may be a probe binding to a GC allele (GC*2: SEQ ID NO: 2, GC*1F: SEQ ID NO: 5 or GC*1S: SEQ ID NO: 6) or a fragment thereof. In this context, a probe is preferably 20 to 30 nucleotides in length. Examples for probes suitable for performing the method described and provided herein are those hybridizing, preferably under stringent conditions, to the nucleotide molecules of the respective GC allele. Such a probe may be further coupled to a detection marker such as FITC, TRITC, Texas Red, Cy-dyes, alexa dyes (Bioprobes), and the like. As further described herein, such a probe may be used, e.g., in a microarray or Northern blot assay or the like to determine the presence of a transcription product such as mRNA of a GC*1F, GC*1S, or GC*2 allele or in a Southern blot or FISH assay or the like to determine the presence of a GC*1F, GC*1S, or GC*2 allele.

The term “antibody” as used herein is to be construed in its broadest sense and specifically encompasses intact monoclonal antibodies, polyclonal antibodies, specific antibodies, multispecific antibodies (e.g., bispecific antibodies) formed from at least two intact antibodies, and antibody fragments, as long as they exhibit the desired biological activity of binding to a particular epitope. An antibody in context with the present invention may specifically bind to GC*2 allele product or fragment thereof, GC*2 allele or fragment thereof, or transcription product (e.g., mRNA) of GC*2 allele or fragment thereof (i.e. of the transcription product). Furthermore, in context of GC*1F and/or GC*1S allele, an antibody specifically bind to GC*1F and/or GC*1S allele product or fragment thereof, GC*1F and/or GC*1S allele or fragment thereof, or transcription product (e.g., mRNA) of GC*2 allele or fragment thereof (i.e. of the transcription product). In context with the present invention, an antibody is able to distinguish between GC*1F (allele, allele product, transcription product of GC*1F allele, or corresponding fragments thereof) and/or GC*1S (allele, allele product, transcription product of GC*1S allele, or corresponding fragments thereof) on the one hand and GC*2 (allele, allele product, transcription product of GC*2 allele, or corresponding fragments thereof) on the other hand. The term “antibody” may refer to any immunologic binding agent such as IgG, IgM, IgA, IgD and IgE. Generally, IgG and/or IgM are preferred because they are most easily made in a laboratory setting. The term “antibody” is used herein to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (see, e.g., Antibodies: A Laboratory Manual, Cold Spring Harbor Laboratory, 1988). Encompassed are chimeric, single chain, and humanized antibodies, human antibodies as well as CDR-grafted antibodies. Also encompassed by the term “antibody” as used herein are “mini-antibodies” or “minibodies”. Minibodies are sFv polypeptide chains which include oligomerization domains at their C-termini, separated from the sFv by a hinge region (Pack, Biochem (1992), 31:1579-1584). The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art (Pack, loc cit; Cumber, Immunology (1992), 149B: 120-126). The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be constructed as requiring production of the antibody by any particular method. For example, monoclonal antibodies to be used in accordance with the present invention may be made by the hybridoma method first described by Kohler, G. et al., Nature 256 (1975) 495, or may be made by recombinant DNA methods (see, e.g., U.S. Pat. No. 4,816,567). “Antibody fragments” comprise a portion of an intact antibody. In context of this invention, antibodies specifically recognize CPS or artificial chimeric CPS obtainable by the in vitro method described herein. In context with the present invention, antibodies may further be coupled to marker molecules. Examples for such marker molecules are enzymes like horse radish peroxidase, alkaline phosphatatase or fluorescent dyes excited and emitting at UV/VIS or infrared wavelengths like FITC, TRITC, Texas Red, Cy-dyes, alexa dyes, etc. As further described herein, antibodies may be used, e.g., in immunological assays such as RIA (Radio Immuno Assay), sandwich (immunometric assay), Western blot, IRMA (Immune Radioimmunometric Assay), EIA (Enzyme Immuno Assay), ELISA (Enzyme Linked Immuno Assay), FIA (Fluorescent Immuno Assay), or CLIA (Chemioluminescent Immune Assay), 2D-Gel, MALDI/TOF, or the like to determine the presence of a GC*1F, GC*1S, or GC*2 allele or a GC*1F, GC*1S, or GC*2 allele product or allele or transcription product (e.g., mRNA) thereof. As a non-limiting example, an antibody specifically binding to the GC*2 allele may be generated using synthetic short peptides. This may, e.g., involve synthesizing short peptide sequences, coupling them to a large carrier molecule, and immunizing the animal of choice such as rabbits mice, goat, etc. with the peptide-carrier molecule. A good response to the desired peptide may be generated with careful selection of the sequence and coupling method. As already mentioned, for further description on antibody production see, e.g., Antibodies: A Laboratory Manual, Cold Spring Harbor Laboratory, 1988. The second step may be the development of a sandwich ELISA as known in the art and also described and exemplified herein below which would be prepared to test for the concentration of, e.g., GC*2 allele product.

The term “hybridization” or “hybridizing” as used herein means any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (for example, C₀ₜ or R₀ₜ analysis) or formed between one nucleic acid sequence present in solution and another nucleic acid sequence immobilized on a solid support (for example, paper, membranes, filters, chips, pins or glass slides, or any other appropriate substrate to which cells or their nucleic acids have been fixed). The term “stringent conditions” and “highly stringent conditions” refers to conditions that permit hybridization between polynucleotides and the claimed polynucleotides. Stringent conditions can be defined by salt concentration, the concentration of organic solvent, for example, formamide, temperature, and other conditions well known in the art. In particular, reducing the concentration of salt, increasing the concentration of formamide, or raising the hybridization temperature can increase stringency. A non-limiting example of “stringent hybridization conditions” is 1×SSC, 0.1% SDS at 65° C. for both, hybridization and wash. A non-limiting example of “highly stringent conditions” is 0.1×SSC, 0.1% SDS at 65° C. for both, hybridization and wash. The term “standard hybridization conditions” refers to salt and temperature conditions substantially equivalent to 5×SSC and 65° C. for both, hybridization and wash. The person skilled in the art will appreciate that such “standard hybridization conditions”, but also “stringent” and “highly stringent hybridization conditions”, are dependent on particular conditions including the concentration of sodium and magnesium in the buffer, nucleotide sequence length and concentration, percent mismatch, percent formamide, and the like. Also important in the determination of “standard”, “stringent” and “highly stringent hybridization conditions” is whether the two sequences hybridizing are RNA-RNA, DNA-DNA or RNA-DNA. Standard hybridization conditions are easily determined by one skilled in the art according to well known formulae, wherein hybridization is typically 10 to 20° C. below the predicted or determined Tₘ with washes of higher stringency, if desired.

The practice of the present invention employs, unless otherwise indicated, conventional techniques of molecular biology, microbiology, recombinant DNA, and so forth which are within the ordinary skill of the art. Such techniques are explained fully in the literature, see, e.g., Sambrook, Fritsch, and Maniatis, Molecular Cloning: A Laboratory Manual, Second Edition (1989), Oligonucleotide Synthesis (M. J. Gait Ed., 1984), Animal Cell Culture (R. I. Freshney, Ed., 1987), the series Methods in Enzymiology (Academic Press, Inc.); Gene Transfer Vectors for Mammalian Cells (J. M. Miller and M. P. Calos eds. 1987), Handbook of Experimental Immunology, (D. M. Weir and C. C. Blackwell, Eds.), Current Protocols in Molecular Biology (F. M. Ausubel, R. Brent, R. E. Kingston, D. D. Moore, J. G. Siedman, J. A. Smith, and K. Struhl, eds., 1987), Current Protocols in Immunology (J. E. coligan, A. M. Kruisbeek, D. H. Margulies, E. M. Shevach and W. Strober, eds., 1991); Annual Review of Immunology; as well as monographs in journals such as Advances in Immunology.

The means and methods described and provided in the present invention are based on the surprising finding that endometriosis in female subjects is correlated with the presence or abundance of different alleles of the vitamin D-binding protein (DBP), namely GC*1F, GC*1S, and GC*2. As described herein, in context with the present invention, it has been found that the presence of the GC*2 allele product in samples of analyzed women was correlated with the presence of endometriosis. Particularly, all women who expressed the GC*2 allele product, but neither the GC*1F nor the GC*1S allele product, i.e. women who were homozygous for the GC*2 allele, suffered from endometriosis. In contrast, the vast majority of the women (79.2%) of the control group who did not suffer from endometriosis did not show expression of the GC*2 allele product. The remaining 20.8% of the women of the control group showed expression of both, GC*1 allele product (GC*1F+GC*1S) and GC*2 allele product, i.e. they were apparently heterozygous for the GC*2 allele. From that and from other results described herein, it can be concluded that not only the presence of the GC*2 allele product or the GC*2 allele or transcription product thereof is indicative for a susceptibility to, predisposition for, presence of and/or risk of developing or suffering from endometriosis, but also that the absence of a GC*1 allele product or the GC*1 allele (as in women homozygous for the GC*2 allele) or transcription product thereof is indicative for an even higher susceptibility to, predisposition for, presence of, and/or risk of developing or suffering from endometriosis.

Accordingly, the methods described herein are in principal based on determining the presence of the GC*2 allele or GC*2 allele product in a biological sample of the female subject to be diagnosed. In one embodiment of the present invention, not only the presence of the GC*2 allele, transcript thereof or the GC*2 allele product, but also the presence of a GC*1 allele (GC*1F or GC*1S), transcript thereof or a GC*1 allele product (GC*1F or GC*1S) is determined. That is, with regard to a GC allele product, the basis of the present invention is to determine whether there is a threonine (Thr, T) (GC*2 allele product) or a lysine (Lys, K) (GC*1F or GC*1S allele products) at position 420 (corresponding to SEQ ID NOs. 1, 3 or 4, respectively) of the DBP contained in the biological sample of the female subject. This can be determined by several methods known in the art and also described and exemplified herein. In other words, with regard to a GC allele product, the present invention is based on the difference of the amino acid residue at position 420 of the GC*1 allele products and the GC*2 allele product. In the GC*1 allele products (SEQ ID NO: 3 for GC*1F and SEQ ID NO: 4 for GC*1S, respectively), there is a threonine (Thr, T) at position 420. In the GC*2 allele product (SEQ ID NO: 1), there is a lysine (Lys, K) at position 420. Similarly, the skilled person can differentiate between the GC*1F and the GC*1S allele products by determining the amino acid at position 416 of SEQ ID NOs. 3 and 4, respectively. In the GC*1F allele product, there is an asparagine (D, Asp) at position 416 of SEQ ID NO: 3. In the GC*1S allele product, there is a glutamate (E, Glu) at position 416 of SEQ ID NO: 4. Analogously, with regard to a GC allele or a transcript such as an mRNA thereof, the present invention bases on determining whether there is an A (GC*2 allele) or a C (GC1F and GC*1S alleles) at position 1565 (corresponding to SEQ ID NOs. 2, 5 or 6, respectively) of the DBP gene or transcript thereof contained in the biological sample of the female subject. This can be determined by several methods known in the art and also described and exemplified herein. In other words, with regard to a GC allele or transcript thereof, the present invention is based on the difference of the nucleic acid residue at position 1565 of the GC*1 alleles and the GC*2 alleles. In the GC*1 alleles (SEQ ID NO: 5 for GC*1F and SEQ ID NO: 6 for GC*1S, respectively), there is a C at position 1565. In the GC*2 allele (SEQ ID NO: 2), there is an A at position 1565. Similarly, the skilled person can differentiate between the GC*1F and the GC*1S alleles by determining the nucleic acid at position 1554 of SEQ ID NOs. 5 and 6, respectively. In the GC*1F allele, there is a T at position 1554 of SEQ ID NO: 5. In the GC*1S allele, there is a G at position 1554 of SEQ ID NO: 6. Consequently, in a preferred embodiment of the present invention, the step of determining the presence of the GC*2 allele product (or the GC*1, the GC*1F, the GC*1S allele product, respectively) or a fragment thereof comprises the determination whether there is a threonine (Thr, T) or a lysine (Lys, K) at position 420 of the respective allele product sequence or at a corresponding position of the respective fragment sequence, based on the amino acid sequence shown in SEQ ID NO: 1 (GC*2 allele product), SEQ ID NO: 3 (GC*1F allele product) or SEQ ID NO: 4 (GC*1S allele product). Analogously, the step of determining the presence of the GC*2 allele (or the GC*1 alleles GC*1F and GC*1S, respectively) or a fragment thereof comprises the determination whether there is an A (GC*2) or a C (GC*1F and GC*1S) at position 1565 of the respective allele or transcript sequence or at a corresponding position of the respective fragment sequence, based on the nucleic acid sequence shown in SEQ ID NO: 2 (GC*2 allele), SEQ ID NO: 5 (GC*1F allele) or SEQ ID NO: 6 (GC*1S allele).

In a specific embodiment, an abundance or expression of the DBP GC*2 allele (transcription) product which is at least about 1.5-fold, preferably at least about 2-fold, more preferably at least about 2.5-fold and even more preferably at least about 3-fold higher as compared to a (healthy) control/control pool is indicative for an increased susceptibility to, predisposition for and/or risk of developing or suffering from endometriosis and/or for the presence of endometriosis.

In another specific embodiment, an abundance or expression of the DBP GC*1S and/or 1F allele (transcription) product which is at least about 1.5-fold, preferably at least about 2-fold, more preferably at least about 2.5-fold and even more preferably at least about 3-fold lower as compared to a (healthy) control/control pool is indicative for an increased susceptibility to, predisposition for and/or risk of developing or suffering from endometriosis and/or for the presence of endometriosis.

Furthermore, generally, as the skilled person readily understands, a transcription product such as an mRNA of a corresponding GC allele can be identified by a particular nucleic acid residue at a particular position within the transcription product sequence which corresponds to the particular nucleic acid residue at the particular position of the corresponding GC allele sequence. Accordingly, in context with the present invention, when it is referred to the determination of the presence of a GC allele or a GC allele product in context of the means and methods provided herein, also a transcription product such as an mRNA of the respective GC allele can be determined and the same conclusions can be drawn analogously. That is, if the presence of a transcription product such as an mRNA of a particular GC allele is determined in a given biological sample of a female subject to be tested, the same conclusions with regard to endometriosis can be drawn as if the presence of the corresponding QG allele or the corresponding GC allele product was determined.

Methods for determining the presence of or detecting a polypeptide or a polynucleotide in a sample are well known in the art and also described and exemplified herein. For example, a polypeptide such as a GC allele product or fragment thereof may be detected by methods such as RIA (Radio Immuno Assay), sandwich (immunometric assay), Western blot, IRMA (Immune Radioimmunometric Assay), EIA (Enzyme Immuno Assay), ELISA (Enzyme Linked Immuno Assay), FIA (Fluorescent Immuno Assay), CLIA (Chemioluminescent Immune Assay), 2D-Gel, MALDI/TOF or the like. As a non-limiting example, in context of the present invention, a sandwich ELISA for determining the presence of a GC*2 allele product (or fragment thereof) may be performed as follows:

Preparing a surface to which a known quantity of capture antibody is bound (in this case, antibody against GC*2 allele product), followed by blocking any non specific binding sites on the surface. As a next step, the antigen-containing sample is applied to the plate (serum). It follows washing the plate, so that unbound antigen is removed. Then, enzyme linked primary antibodies are applied as detection antibodies which also bind specifically to the antigen. Then the plate is washed, so that the unbound antibody-enzyme conjugates are removed, followed by applying a chemical which is converted by the enzyme into a color or fluorescent or electrochemical signal. Finally, the absorbency or fluorescence of the plate wells is measured to determine the presence and quantity of antigen (GC*2 allele product). In this context, e.g., a protocol adopted from Jorgensen, Scan J Clin Invest (2004), 64: 157-166, may be performed as follows in order to target the GC*2 allele product: First, 5 mg/mL purified human Gc globulin (SSI, Denmark) is coated in MaxiSorp plates (NUNC, Denmark; 100 mL/well) using 50 mM sodium carbonate (Merck, Germany), pH 9.6 as coating buffer. After three washings of 1 min each using 50 mM Tris (Sigma, USA), 0.3 M NaCl (Merck, Germany), 1% Tween 20 (Merck, Germany), pH 7.5 (TTN), the plate is blocked for 30 min using the same buffer (using the detergent Tween 20 as blocking agent). Pre-incubated (1 h at room temperature) dilutions of a standard Gc globulin preparation (SSI) with known concentration, unknown samples and controls (human plasma pool and Gc globulin) in TTN containing monoclonal mouse-anti-Gc globulin 1:400 (SSI, Denmark) are added and incubated for 1 h. The plate is washed three times with TTN, followed by 1 h of incubation with alkaline phosphatase-conjugated goat-anti-mouse antibody (Sigma, USA). After another three washes, the plate is developed using p-nitrophenyl phosphate (Sigma, USA) 1 mg/mL in 1 M diethanolamine (Sigma, USA), pH 9.8, 0.5 mM MgCl2 (Merck, Germany). The plate is read on a VERSamax turnable microplate reader (Molecular Devises, USA) at 405 nm using background-subtraction at 690 nm. As will be understood by the skilled person, the determination of the presence of a GC*1 allele product (or fragment thereof) can be performed analogously.

In context of detecting a polynucleotide such as a GC allele, for example, methods such as PCR, qPCR, RT-PCR, qRT-PCR, RT-qPCR, sequencing (optionally subsequent to a PCR, qPCR, RT-PCR, qRT-PCR or RT-qPCR), Light Cycler®, TaqMan® Platform and Assays or quantigene assay (Zhou, Anal Biochem (2000), 282: 46-53), FISH, Northern blot, dot blot, microarrays, next generation sequencing (VanGuilder, Biotechniques (2008), 44(5): 619-26; Elvidge, Pharmacogenomics (2006), 7: 123-134; Metzker, Nat Rev Genet (2010), 11: 31-46; Kafatos, NAR (1979), 7: 1541-1552), or the like may be applied. As a non-limiting example, in context of the present invention, a PCR for determining the presence of GC*2 allele may be performed as follows, adopted from Pani, JCEM (2002), 87: 2564-2567:

PCRs are performed in a total volume of 25 μl for each pair of primers containing 50 mM KCl, 1.5 mM MgCl2, 10 mM Tris HCl (pH 8.3), 0.25 mM of each dNTP, 1.2 μM of each primer, and 0.25 U Taq polymerase (Promega Corp., Madison, Wis.) using 100 ng template DNA. As will be understood by the skilled person, the determination of the presence of a GC*1 allele (or fragment thereof) or transcription product (or fragment thereof) can be performed analogously.

All patents, patent applications, and publications mentioned herein, both supra and infra, are hereby incorporated herein by reference.

The invention is further illustrated by the following non-limiting examples.

### EXAMPLES

### Example 1

### Methods

### Analyzed Subjects

Consenting women age 18 to 49 undergoing surgery for pain and/or infertility or elective tubal ligation. Women with acute or chronic medical conditions were excluded.

### Samples

After obtaining written informed consent, serum was collected from reproductive age women with pain and/or infertility who were undergoing laparoscopic evaluation. Venous blood was collected preoperatively, the samples were centrifuged for 10 min at 3.500 g and the supernatant was aliquoted and stored at −70° C. until analysis. Samples were collected at the University of Pennsylvania (Philadelphia, USA) in the years 2003 to 2005 after study approval by the Institutional Review Board. For surgical staging of endometriosis, if present, the Revised Classification of the American Society of Reproductive Medicine (Fertil Steril (1997), 67: 817-821) was used. As a control group, serum from women was used who underwent laparoscopy for tubal ligation (N=23), tubal re-anastamosis (N=2), or elective bilateral oophorectomy (N=1) who were asymptomatic and surgically confirmed to be free of endometriosis. There were no statistical differences in menstrual cycle phase of serum collection, use of oral contraceptives or progesterone, or GnRH analogs between groups.

### Pools

Samples from each disease stage were randomly selected and equal aliquots pooled together. Four serum pools were thus investigated regarding differences in protein abundance: Pool I/II (minimal/mild, N=20), III (moderate, N=16), IV (severe, N=20) and the control pool (N=20).

### Depletion of Serum Pools

A commercially-available multiple affinity spin column MARS Hu-14 (Agilent Technologies) was used for the depletion of 14 most highly abundant proteins from the serum pools.

100 μl of serum sample were mixed with 100 μl wash buffer (Agilent Technologies) containing additionally 2% Chaps and 8 M Urea. The sample was kept for 15 min at room temperature and filtered through 0.22 μm spin filter. The 200 μl sample was diluted to 2 ml with wash buffer containing 0.66 M Urea to obtain a concentration of 0.1% CHAPS and 1 M Urea. For depletion, the cartridge was placed into a 1.5 ml tube. 200 μl of diluted sample was filled into the spin column and centrifuged for 1 min at 100×g, then removed and kept for 5 min at room temperature. Threefold washing of the column with 350 μl wash buffer containing 1 M urea followed, with centrifugation for 2.5 min at 100×g per wash step. The flow-trough was collected and stored at −20° C.

### Sample Concentration and Protein Quantification

Centrifugal concentrators Vivaspin 2 (Sartorius Stedim Biotech) with a MWCO 2.000 Da were used. After concentration, the samples were washed three times in the Centrifugal concentrator with 30 mM Tris-HCl buffer at pH 8.5. A protein assay based on the method of Bradford (Sigma Aldrich, Vienna, Austria) was used for protein quantification in order to determine the sample volume needed for the subsequent isoelectric focusing.

### Protein Labeling

To facilitate the inter-pool and inter-gel comparisons, the CyDye fluorescence dyes Cy2, Cy3 and Cy5 (GE Healthcare) were used. Each dye has different absorption and emission spectra allowing for differentiation between multiple samples on the same 2D-Gel. The labeling procedure was undertaken according to the instruction manual from GE Healthcare for Amersham CyDye DIGE Flours (minimal dyes). 25 μg of each depleted sample was diluted in lysis buffer and labeled with 170 pmol CyDye fluorescence dye. The depleted samples were run in multiple permutations on multiple gels. Each gel was comprised of one internal standard (made by mixing equal amounts of protein from each pool) always labeled with Cy2 and two different pools each with Cy3 and Cy5. Each of the four pools was separated four times, twice labeled with Cy 3 and twice with Cy 5. This cross-labeling equalizes differences in fluorescence intensity among the three fluors and the use of an internal standard allows for the accurate detection of small differences in protein abundance between pools, while minimizing inter-gel variations.

### Isoelectric Focusing

For the separation of proteins in the first dimension, 18 cm IPG-strips (GE Healthcare) with a non linear range from pH 3-11 were used. Isoelectric focusing and equilibration of the IPG-strips before SDS-PAGE was undertaken as previously described (Kienzl, Transplantation (2009), 88: 553-560).

### SDS-PAGE

For separation in the second dimension, 10-20% Tris-Tricine gradient gels was used as previously described by Schägger (Nature Protoc (2006), 1: 16-22). For SDS-PAGE, an Ettan DALTtwelve Separation Unit (GE Healthcare) was used. The electrophoresis was started with 1 W per Gel for 1 h then increased to a power of 8-12 W per gel.

Imaging and Analysis

The gel imaging was carried out as previously described (Kienzl, loc cit).

### Statistical Analysis

In order to minimize false positive results, the following criteria were pre-determined:

(1) Inter-group differences between protein spot abundance had to exceed a factor of +/−2.0. (2) The p-value of the corresponding equal variance two-tailed student's t-test had to be lower than 0.05. To pare down the number of potentially interesting spots, focus was laid on those which either increased with severity of disease or decreased with disease severity—as these were more inclined to be clinically meaningful. All spots that fit these criteria were marked as potentially interesting and were picked out of gels for further identification.

### Mass Spectrometric Analysis

Spot picking, protein excision, and in-gel digestion were carried as previously described (Sobieszek, Arch Biochem Biophys (2006), 454: 197-205) using trypsin from porcine pancreas (Sigma Aldrich, Vienna, Austria) for the standard identification and chymotrypsin from bovine pancreas (Sigma Aldrich, Vienna, Austria) for identification of DBP allele products.

Protein digests were analyzed using MALDI TOF/TOF 4800 plus Analyzer (Applied Biosystems) for standard identification and nanoLC-ESI-MS (LTQ Orbitrap XL) (ThermoScientific) equipped with a nanospray interface and coupled to an UltiMate 3000 system (Dionex) for identification of DBP allele products. MS/MS spectra were searched against human database using Mascot (Matrix Science) and Sequest (ThermoFinnigan) algorithm. For Mascot search (standard identification), NCBInr database for Homo sapiens were used. Protein scores >63 indicate identity or extensive homology (p<0.05). For Sequest search, the identified peptides were further evaluated using charge state versus cross-correlation number (Xcorr) as previously described (Sobieszek, loc cit). The criteria for positive identification of peptides were Xcorr>1.5 for singly charged ions, Xcorr>2.0 for doubly charged ions, and Xcorr>2.5 for triply charged ions. Fixed modification was carbamidomethylation of Cys residues, variable modifications were Met oxidation and neutral loss on Ser and Thr residues of NeuNAc-Hex-HexNAc (656.6 Da) and Hex-HexNAc (365.4 Da).

### Validation of DBP Allele Products in Single Specimens

The validation of DBP allele products in single samples was accomplished by separation on gels as described above, with the following modifications. Single samples were depleted using ProteoPrep Blue Albumin and IgG Depletion Kit (Sigma Aldrich, Vienna, Austria). 81 single samples were analyzed: 20 with disease stage I (minimal); 20 with stage II (mild); 17 with stage IV (severe) and 24 control samples. For isoelectric focusing, 18 cm IPG-strip with a non linear range from pH 3-5.6 were used. Previous to isoelectric focusing, 25 μg of each depleted sample was labeled with one of the CyDye dyes and investigated once on 2D-PAGE.

DBP forms a chain of 3 spots when separating in 2D-Gels due to differences in amino acid sequence and glycosylation of the allelic forms. The allele products found in single samples could be classified according to the pattern of the respective spots on the 2D-Gel. For confirmation, the volumes of the respective spots containing the DBP allele products were calculated using DeCyder Differential Analysis Software. The relative ratio of the spots containing GC*2 and GC*1F/GC*1S was calculated for relative quantification of the DBP allele products present in the sample. The differences in expression of allele products of single specimens were compared between groups using Chi-square (GraphPad Prism, Version 4).

### Example 2

### Results

To investigate differences in abundance of serum proteins in women with and without endometriosis, 2D-DIGE comparisons were performed between women of different disease stages. After serum depletion, the proteins of the stage-specific pools were labeled with CyDye dyes, separated using 2D-PAGE, in-gel digestion with trypsin and identified using mass spectrometry. Each pooled sample was analyzed four times by running it on four separate gels. We found 25 protein spots with a significant difference in abundance between diseased and disease-free subjects (FIG. 1): 12 spots showed an increase in abundance with severity of endometriosis, 13 spots a decrease in abundance with severity. 13 different proteins could be identified in the 25 spots using MALDI TOF/TOF mass spectrometry. A series of important acute phase proteins and complement components was differentially abundant in serum of women with endometriosis (Table 1).

TABLE 1
Proteins identified with corresponding abundance change in serum pools with the severity of endometriosis.

| | | | | | | | | | Total | | | |
|---|---|---|---|---|---|---|---|---|---|---|---|---|
| Spot | | I + II/Control | | III/Control | | IV/Control | | UniProt | Ion | Protein | Molecular | |
| No. | Protein identified | Av. Ratio | p-value | Av. Ratio | p-value | Av. Ratio | p-value | acc. no. | score | score | mass [kDa] | pI |
| 1 | Complement factor B | −1.44 | 0.18 | 1.07 | 0.86 | 2.5 | 0.0057 | P00751 | 66 | 107 | 83.0 | 6.66 |
| 2 | Alpha-1-antitrypsin | −1.09 | 0.68 | 2.58 | 0.026 | 2.97 | 0.013 | P01009 | 126 | 170 | 44.3 | 5.37 |
| 3 | Alpha-1-antitrypsin | −1.03 | 0.83 | 2.25 | 0.051 | 2.7 | 0.023 | P01009 | 638 | 816 | 44.3 | 5.37 |
| 4 | Vitamin D-binding | 3.11 | 0.018 | 2.77 | 0.022 | 2.97 | 0.017 | P02774 | 1010 | 1.170 | 51.2 | 5.22 |
| | protein (DBP) | | | | | | | | | | | |
| 5 | Complement factor B | 1.12 | 0.59 | −1.11 | 0.63 | 2.48 | 0.01 | P00751 | 162 | 186 | 83.0 | 6.66 |
| | Complement factor | | | | | | | Q03591 | 120 | 143 | 35.7 | 7.10 |
| | H-related protein 1 | | | | | | | | | | | |
| 6 | Complement factor | −1.15 | 0.46 | −2.79 | 0.0049 | −2.1 | 0.017 | Q03591 | 78 | 117 | 35.7 | 7.10 |
| | H-related protein 1 | | | | | | | | | | | |
| 7 | Serum albumin | −1.34 | 0.24 | 2.01 | 0.17 | 2.22 | 0.036 | P02768 | 101 | | 66.5 | 5.67 |
| | Complement C4-A, | | | | | | | P0C0L4/ | 94 | | 156.4 | 6.29/ |
| | C4-B | | | | | | | P0C0L5 | | | | 6.36 |
| 8 | Complement C4-A, | −1.16 | 0.65 | 2.66 | 0.099 | 2.83 | 0.0068 | P0C0L4/ | 117 | 155 | 156.4 | 6.29/ |
| | C4-B | | | | | | | P0C0L5 | | | | 6.36 |
| 9 | Serum albumin | −1.11 | 0.66 | 3.06 | 0.14 | 2.53 | 0.017 | P02768 | 251 | 290 | 66.5 | 5.67 |
| | Complement C4-A, | | | | | | | P0C0L4/ | 80 | 111 | 156.4 | 6.29/ |
| | C4-B | | | | | | | P0C0L5 | | | | 6.36 |
| 10 | Alpha-1-antitrypsin | −1.07 | 0.68 | 1.23 | 0.63 | 2.25 | 0.025 | P01009 | 161 | 217 | 44.3 | 5.37 |
| 11 | Inter-alpha-trypsin | −1.82 | 0.037 | −7.35 | 0.0011 | −3.14 | 0.003 | Q14624 | 231 | 268 | 100.3 | 6.11 |
| | inhibitor heavy | | | | | | | | | | | |
| | chain H4 | | | | | | | | | | | |
| 12 | Complement C4-A, | 1.03 | 0.72 | 5.57 | 0.0047 | 5.36 | 0.0032 | P0C0L4/ | 457 | 554 | 156.4 | 6.29/ |
| | C4-B | | | | | | | P0C0L5 | | | | 6.36 |
| 13 | Complement C3 | −1.13 | 0.56 | −3.26 | 0.00042 | −1.46 | 0.15 | P01024 | 586 | 628 | 185.0 | 6.00 |
| 14 | Complement C3 | 1.11 | 0.9 | −14.28 | 0.00004 | −4.63 | 0.0014 | P01024 | 276 | 298 | 185.0 | 6.00 |
| 15 | Complement C4-A, | −1.61 | 0.1 | −5.01 | 0.0029 | −2.61 | 0.013 | P0C0L4/ | 353 | 397 | 156.4 | 6.29/ |
| | C4-B | | | | | | | P0C0L5 | | | | 6.36 |
| 16 | Ceruloplasmin | −2.02 | 0.031 | −23.51 | 0.00037 | −25.25 | 0.000014 | P00450 | 51 | 69 | 120.1 | 5.41 |
| 17 | Complement C3 | −1.05 | 0.81 | −14.95 | 0.00024 | −4.32 | 0.0034 | P01024 | 209 | 241 | 185.0 | 6.00 |
| 18 | Inter-alpha-trypsin | 1.72 | 0.054 | 2.96 | 0.013 | 3.07 | 0.007 | P19823 | 72 | 111 | 72.5 | 5.75 |
| | Inhibitor heavy | | | | | | | | | | | |
| | chain H2 | | | | | | | | | | | |
| 19 | Haptoglobin | −2.06 | 0.023 | −5.42 | 0.00096 | −2.24 | 0.012 | P00738 | 134 | 168 | 43.3 | 6.13 |
| 20 | Ceruloplasmin | −1.86 | 0.028 | −15.34 | 0.000023 | −8.24 | 0.0000015 | P00450 | 83 | 95 | 120.1 | 5.41 |
| 21 | Vitamin D-binding | −2.68 | 0.0039 | −12.3 | 0.00013 | −7.64 | 0.000028 | P02774 | 72 | 104 | 51.2 | 5.22 |
| | protein (DBP) | | | | | | | | | | | |
| 22 | Alpha-1-antitrypsin | −1.09 | 0.73 | 1.46 | 0.23 | 2.14 | 0.045 | P01009 | 36 | 68 | 44.3 | 5.37 |
| 23 | Alpha-1-antitrypsin | −1.29 | 0.29 | −9.43 | 0.000085 | −5.42 | 0.00071 | P01009 | 143 | 168 | 44.3 | 5.37 |
| 24 | Serum amyloid P | −1.29 | 0.23 | −6.8 | 0.000024 | −2.71 | 0.0091 | P02743 | 68 | 87 | 23.3 | 6.12 |
| | component | | | | | | | | | | | |
| 25 | Hemoglobin subunit | −3.25 | 0.0044 | −2.45 | 0.014 | −2.96 | 0.009 | P69905 | 158 | 223 | 15.1 | 8.73 |
| | alpha | | | | | | | | | | | |

Identified protein, Average ratio, p-values, UniProt accession numbers and Mascot Total Ion scores are listed for spots which differ in spot volume statistically significantly (p-value ≦0.05) by a factor of at least 2 between an endometriosis pool and the control group.
Average ratio and p-values originate from the DeCyder BVA module, representing the mean change in the standardized abundance of 4 independent data points per pool and the corresponding equal variance two-tailed t-test.
Data were bold labeled when the average ratio was higher than 2 and the statistical significance was confirmed. Mascot Protein scores >63 indicate identity or extensive homology (p-value ≦0.05).
Calculated Mw and pI correspond to the secreted protein, without the signal sequence.

The abundance of the entire DBP, found in spot 4 on the gel was higher in all endometriosis pools by a factor of approximately 3 compared to the control pool. It was decided that DBP merited further detailed investigation due to its uniform high abundance in all endometriosis stages and its known action as a macrophage activator.

Since it is commonly known that DBP is a genetic polymorphism in the humans (Cleve, loc cit), the specific allele product present in spot 4 were identified. Therefore, the digestion enzyme was changed from trypsin to chymotrypsin B yielding a peptide from amino acid 411 to 422 (numbered according to the secreted protein, without the signal sequence; cf. SEQ ID NO: 1) that enables the differentiation of the three major allele products (GC*1F, GC*1S, GC*2) and their possible glycosylation state (FIG. 2A). Using nanoLC-ESI-MS, the 1310.55 Da peptide corresponding to 411-KAKLPDATPKEL-422 (Sequest Xcorr score (see Methods in Molecular Biology 367: Mass Spectrometry Data Analysis in Proteomics; R. Matthiesen ed.; Nesuizhskii A. Chapter 6: Protein Identification by Tandem Mass Spectrometry and Sequence Database Searching, p 87 ff): 2.559) was detected which is the specific amino acid sequence of the DBP GC*2 allele product. The MS/MS spectrum is shown in FIG. 2C.

It has been reported that the three major allele products differ in their isoelectric point (pI) due to alterations in their amino acid sequence and in their possible glycosylation state (Speeckaert, Clin Chim Acta (2006), 372: 33-42). Therefore, it was hypothesized and subsequently confirmed that the other two major DBP allele products (GC*1F and GC*1S) are contained in spots lateral to spot 4 in the 2D-Gel (FIG. 2B). Spot 4a contained the DBP GC*1F allele product as unambiguously identified by the peptide 411-KAKLPDATPTEL-422 (FIG. 2D) (Mw: 1283.72 Da; Xcorr score: 3.565) as well as GC*1S as identified by the peptide 411-KAKLPEATPTEL-422 (Mw 1297.74 Da; Xcorr score: 2.839). Spot 4β likewise contained GC*1F and GC*1S allele products, however, with both proteins glycosylated at the threonine at position 420, accounting for the separation of 4α and 4β. During MS/MS fragmentation, neutral losses of 291.13 Da (NeuNAc)₁, 453.13 Da (NeuNAc)₁(Gal₁) and 656.33 (NeuNAc)₁(Gal)₁(GalNAc)₁ were observed which indicated that the GC*1F (FIG. 2E) and GC*1S specific peptides in spot 4β were modified with a linear (NeuNAc)₁-(Gal)₁-(GalNAc)₁ trisaccharide, as previously described (Borges, J Proteome Res (2008), 7: 4143-4153).

The observation that DBP is seen as 3 separate spots with differing pI values, as described by Arnaud et al. (Arnaud, Hum Genet (1993), 92: 183-188), can be explained by two factors. First, the GC*2 allele product in spot 4 has a higher pI (pI 5.1) than the GC*1 variants in spot 4a (pI 4.94 and 4.95) because of the exchange from threonine (Thr, T) to lysine (Lys, K) at position 420. Second, the NeuNAc acidifies trisaccharide glycosylated GC*1 allele products resulting in a third distinct spot, 4β (pI 4.84 and 4.85), on the 2D gel. Because of the almost equivalent pI of GC*1F and GC*1S, it was not possible to differentiate between those two allele products on the basis of 2D gels.

The electrophoretic separation and subsequent identification of the allele products allowed to determine that only GC*2 allele product showed difference between endometriosis pools and the control group, as previously discussed (FIG. 3). However, the spots 4α and 4β containing GC*1 allele products did not show significant differences in expression between any pools, nor did the total amount of DBP (combined density of spots 4, 4α and 4β). This is consistent with the findings of the study by Borkowski et al. (Borkowski, Postepy Hig Dosw (2008), 62: 103-109).

The subsequent aim was to validate the allele products of DBP in single serum samples. The classification of the allele products was performed according to the pattern of spots on the 2D gel. Three combinations were observed: Either just the GC*1 allele products were present (spot 4α and 4β) (FIG. 4A), or allele products GC*1 and GC*2 were present (all three spots) (FIG. 4B), or only the GC*2 allele product (spot 4) (FIG. 4C).

In those cases when allele products GC*1 and GC*2 were present, their relative ratio to each other was evaluated to verify whether differences between disease stages occur. For calculation, the ratio of spot volume 4 to 4α was used, and the volume of spot 4β was not included in the calculation as it is overlaid by a small alpha-1-antitrypsin spot. The mean relative ratio of spot 4 to spot 4α in all single samples expressing allele products GC*1 and GC*2 was 1.20 with a standard deviation of +/−0.45 (FIG. 6). No statistically significant difference between disease stages and the control group was observed. Therefore, we considered all samples which contained both allele products to be a homogeneous group with equal expression of the two alleles.

As depicted in FIG. 5, the distribution of allele products among single specimens was significantly different between the groups evaluated (stage I/II, stage IV, and control), p=0.006. GC*2 allele product appeared more frequently in the endometriosis samples than in the control group. Of note, no subject in the control group expressed the GC*2 allele product alone, whereas in endometriosis subjects the expression of GC*2 alone was observed in 17.5%. Conversely, 79.2% of the single specimens in the control pool expressed GC*1 allele product alone compared to 50% of stage I and II and 23.5% of stage IV, respectively.

Sequences

SEQ ID NO: 1
GC*2
human aa sequence
LERGRDYEKNKVCKEFSHLGKEDFTSLSLVLYSRKFPSGTFEQVSQLVKE
VVSLTEACCAEGADPDCYDTRTSALSAKSCESNSPFPVHPGTAECCTKEG
LERKLCMAALKHQPQEFPTYVEPTNDEICEAFRKDPKEYANQFMWEYSTN
YGQAPLSLLVSYTKSYLSMVGSCCTSASPTVCFLKERLQLKHLSLLTTLS
NRVCSQYAAYGEKKSRLSNLIKLAQKVPTADLEDVLPLAEDITNILSKCC
ESASEDCMAKELPEHTVKLCDNLSTKNSKFEDCCQEKTAMDVFVCTYFMP
AAQLPELPDVELPTNKDVCDPGNTKVMDKYTFELSRRTHLPEVFLSKVLE
PTLKSLGECCDVEDSTTCFNAKGPLLKKELSSFIDKGQELCADYSENTFT
EYKKKLAERLKAKLPDATPKELAKLVNKRSDFASNCCSINSPPLYCDSEI
DAELKNIL
SEQ ID NO: 3
GC*1S
human aa sequence
LERGRDYEKNKVCKEFSHLGKEDFTSLSLVLYSRKFPSGTFEQVSQLVKE
VVSLTEACCAEGADPDCYDTRTSALSAKSCESNSPFPVHPGTAECCTKEG
LERKLCMAALKHQPQEFPTYVEPTNDEICEAFRKDPKEYANQFMWEYSTN
YGQAPLSLLVSYTKSYLSMVGSCCTSASPTVCFLKERLQLKHLSLLTTLS
NRVCSQYAAYGEKKSRLSNLIKLAQKVPTADLEDVLPLAEDITNILSKCC
ESASEDCMAKELPEHTVKLCDNLSTKNSKFEDCCQEKTAMDVFVCTYFMP
AAQLPELPDVELPTNKDVCDPGNTKVMDKYTFELSRRTHLPEVFLSKVLE
PTLKSLGECCDVEDSTTCFNAKGPLLKKELSSFIDKGQELCADYSENTFT
EYKKKLAERLKAKLPEATPTELAKLVNKRSDFASNCCSINSPPLYCDSEI
DAELKNIL
SEQ ID NO: 4
GC*1F
human aa sequence
LERGRDYEKNKVCKEFSHLGKEDFTSLSLVLYSRKFPSGTFEQVSQLVKE
VVSLTEACCAEGADPDCYDTRTSALSAKSCESNSPFPVHPGTAECCTKEG
LERKLCMAALKHQPQEFPTYVEPTNDEICEAFRKDPKEYANQFMWEYSTN
YGQAPLSLLVSYTKSYLSMVGSCCTSASPTVCFLKERLQLKHLSLLTTLS
NRVCSQYAAYGEKKSRLSNLIKLAQKVPTADLEDVLPLAEDITNILSKCC
ESASEDCMAKELPEHTVKLCDNLSTKNSKFEDCCQEKTAMDVFVCTYFMP
AAQLPELPDVELPTNKDVCDPGNTKVMDKYTFELSRRTHLPEVFLSKVLE
PTLKSLGECCDVEDSTTCFNAKGPLLKKELSSFIDKGQELCADYSENTFT
EYKKKLAERLKAKLPDATPTELAKLVNKRSDFASNCCSINSPPLYCDSEI
DAELKNIL
SEQ ID NO: 2
GC*2
human DNA sequence
TTTAATAATAATTCTGTGTTGCTTCTGAGATTAATAATTGATTAATTCAT
AGTCAGGAATCTTTGTAAAAAGGAAACCAATTACTTTTGGCTACCACTTT
TACATGGTCACCTACAGGAGAGAGGAGGTGCTGCAAGACTCTCTGGTAGA
AAAATGAAGAGGGTCCTGGTACTACTGCTTGCTGTGGCATTTGGACATGC
TTTAGAGAGAGGCCGGGATTATGAAAAGAATAAAGTCTGCAAGGAATTCT
CCCATCTGGGAAAGGAGGACTTCACATCTCTGTCACTAGTCCTGTACAGT
AGAAAATTTCCCAGTGGCACGTTTGAACAGGTCAGCCAACTTGTGAAGGA
AGTTGTCTCCTTGACCGAAGCCTGCTGTGCGGAAGGGGCTGACCCTGACT
GCTATGACACCAGGACCTCAGCACTGTCTGCCAAGTCCTGTGAAAGTAAT
TCTCCATTCCCCGTTCACCCAGGCACTGCTGAGTGCTGCACCAAAGAGGG
CCTGGAACGAAAGCTCTGCATGGCTGCTCTGAAACACCAGCCACAGGAAT
TCCCTACCTACGTGGAACCCACAAATGATGAAATCTGTGAGGCGTTCAGG
AAAGATCCAAAGGAATATGCTAATCAATTTATGTGGGAATATTCCACTAA
TTACGGACAAGCTCCTCTGTCACTTTTAGTCAGTTACACCAAGAGTTATC
TTTCTATGGTAGGGTCCTGCTGTACCTCTGCAAGCCCAACTGTATGCTTT
TTGAAAGAGAGACTCCAGCTTAAACATTTATCACTTCTCACCACTCTGTC
AAATAGAGTCTGCTCACAATATGCTGCTTATGGGGAGAAGAAATCAAGGC
TCAGCAATCTCATAAAGTTAGCCCAAAAAGTGCCTACTGCTGATCTGGAG
GATGTTTTGCCACTAGCTGAAGATATTACTAACATCCTCTCCAAATGCTG
TGAGTCTGCCTCTGAAGATTGCATGGCCAAAGAGCTGCCTGAACACACAG
TAAAACTCTGTGACAATTTATCCACAAAGAATTCTAAGTTTGAAGACTGT
TGTCAAGAAAAAACAGCCATGGACGTTTTTGTGTGCACTTACTTCATGCC
AGCTGCCCAACTCCCCGAGCTTCCAGATGTAGAGTTGCCCACAAACAAAG
ATGTGTGTGATCCAGGAAACACCAAAGTCATGGATAAGTATACATTTGAA
CTAAGCAGAAGGACTCATCTTCCGGAAGTATTCCTCAGTAAGGTACTTGA
GCCAACCCTAAAAAGCCTTGGTGAATGCTGTGATGTTGAAGACTCAACTA
CCTGTTTTAATGCTAAGGGC
CCTCTACTAAAGAAGGAACTATCTTCTTTCATTGACAAGGGACAAGAACT
ATGTGCAGATTATTCAGAAAATACATTTACTGAGTACAAGAAAAAACTGG
CAGAGCGACTAAAAGCAAAATTGCCTGATGCCACACCCACGGAACTGGCA
AAGCTGGTTAACAAGCACTCAGACTTTGCCTCCAACTGCTGTTCCATAAA
CTCACCTCCTCTTTACTGTGATTCAGAGATTGATGCTGAATTGAAGAATA
TCCTGTAGTCCTGAAGCATGTTTATTAACTTTGACCAGAGTTGGAGCCAC
CCAGGGGAATGATCTCTGATGACCTAACCTAAGCAAAACCACTGAGCTTC
TGGGAAGACAACTAGGATACTTTCTACTTTTTCTAGCTACAATATCTTCA
TACAATGACAAGTATGATGATTTGCTATCAAAATAAATTGAAATATAATG
CAAACCATAAAAAAAAAAAAAAAAAAAAAAA
SEQ ID NO: 5
GC*1F
human DNA sequence
TTTAATAATAATTCTGTGTTGCTTCTGAGATTAATAATTGATTAATTCAT
AGTCAGGAATCTTTGTAAAAAGGAAACCAATTACTTTTGGCTACCACTTT
TACATGGTCACCTACAGGAGAGAGGAGGTGCTGCAAGACTCTCTGGTAGA
AAAATGAAGAGGGTCCTGGTACTACTGCTTGCTGTGGCATTTGGACATGC
TTTAGAGAGAGGCCGGGATTATGAAAAGAATAAAGTCTGCAAGGAATTCT
CCCATCTGGGAAAGGAGGACTTCACATCTCTGTCACTAGTCCTGTACAGT
AGAAAATTTCCCAGTGGCACGTTTGAACAGGTCAGCCAACTTGTGAAGGA
AGTTGTCTCCTTGACCGAAGCCTGCTGTGCGGAAGGGGCTGACCCTGACT
GCTATGACACCAGGACCTCAGCACTGTCTGCCAAGTCCTGTGAAAGTAAT
TCTCCATTCCCCGTTCACCCAGGCACTGCTGAGTGCTGCACCAAAGAGGG
CCTGGAACGAAAGCTCTGCATGGCTGCTCTGAAACACCAGCCACAGGAAT
TCCCTACCTACGTGGAACCCACAAATGATGAAATCTGTGAGGCGTTCAGG
AAAGATCCAAAGGAATATGCTAATCAATTTATGTGGGAATATTCCACTAA
TTACGGACAAGCTCCTCTGTCACTTTTAGTCAGTTACACCAAGAGTTATC
TTTCTATGGTAGGGTCCTGCTGTACCTCTGCAAGCCCAACTGTATGCTTT
TTGAAAGAGAGACTCCAGCTTAAACATTTATCACTTCTCACCACTCTGTC
AAATAGAGTCTGCTCACAATATGCTGCTTATGGGGAGAAGAAATCAAGGC
TCAGCAATCTCATAAAGTTAGCCCAAAAAGTGCCTACTGCTGATCTGGAG
GATGTTTTGCCACTAGCTGAAGATATTACTAACATCCTCTCCAAATGCTG
TGAGTCTGCCTCTGAAGATTGCATGGCCAAAGAGCTGCCTGAACACACAG
TAAAACTCTGTGACAATTTATCCACAAAGAATTCTAAGTTTGAAGACTGT
TGTCAAGAAAAAACAGCCATGGACGTTTTTGTGTGCACTTACTTCATGCC
AGCTGCCCAACTCCCCGAGCTTCCAGATGTAGAGTTGCCCACAAACAAAG
ATGTGTGTGATCCAGGAAACACCAAAGTCATGGATAAGTATACATTTGAA
CTAAGCAGAAGGACTCATCTTCCGGAAGTATTCCTCAGTAAGGTACTTGA
GCCAACCCTAAAAAGCCTTGGTGAATGCTGTGATGTTGAAGACTCAACTA
CCTGTTTTAATGCTAAGGGC
CCTCTACTAAAGAAGGAACTATCTTCTTTCATTGACAAGGGACAAGAACT
ATGTGCAGATTATTCAGAAAATACATTTACTGAGTACAAGAAAAAACTGG
CAGAGCGACTAAAAGCAAAATTGCCTGATGCCACACCCACGGAACTGGCA
AAGCTGGTTAACAAGCACTCAGACTTTGCCTCCAACTGCTGTTCCATAAA
CTCACCTCCTCTTTACTGTGATTCAGAGATTGATGCTGAATTGACGAATA
TCCTGTAGTCCTGAAGCATGTTTATTAACTTTGACCAGAGTTGGAGCCAC
CCAGGGGAATGATCTCTGATGACCTAACCTAAGCAAAACCACTGAGCTTC
TGGGAAGACAACTAGGATACTTTCTACTTTTTCTAGCTACAATATCTTCA
TACAATGACAAGTATGATGATTTGCTATCAAAATAAATTGAAATATAATG
CAAACCATAAAAAAAAAAAAAAAAAAAAAAA
SEQ ID NO: 6
GC*1S
human DNA sequence
TTTAATAATAATTCTGTGTTGCTTCTGAGATTAATAATTGATTAATTCAT
AGTCAGGAATCTTTGTAAAAAGGAAACCAATTACTTTTGGCTACCACTTT
TACATGGTCACCTACAGGAGAGAGGAGGTGCTGCAAGACTCTCTGGTAGA
AAAATGAAGAGGGTCCTGGTACTACTGCTTGCTGTGGCATTTGGACATGC
TTTAGAGAGAGGCCGGGATTATGAAAAGAATAAAGTCTGCAAGGAATTCT
CCCATCTGGGAAAGGAGGACTTCACATCTCTGTCACTAGTCCTGTACAGT
AGAAAATTTCCCAGTGGCACGTTTGAACAGGTCAGCCAACTTGTGAAGGA
AGTTGTCTCCTTGACCGAAGCCTGCTGTGCGGAAGGGGCTGACCCTGACT
GCTATGACACCAGGACCTCAGCACTGTCTGCCAAGTCCTGTGAAAGTAAT
TCTCCATTCCCCGTTCACCCAGGCACTGCTGAGTGCTGCACCAAAGAGGG
CCTGGAACGAAAGCTCTGCATGGCTGCTCTGAAACACCAGCCACAGGAAT
TCCCTACCTACGTGGAACCCACAAATGATGAAATCTGTGAGGCGTTCAGG
AAAGATCCAAAGGAATATGCTAATCAATTTATGTGGGAATATTCCACTAA
TTACGGACAAGCTCCTCTGTCACTTTTAGTCAGTTACACCAAGAGTTATC
TTTCTATGGTAGGGTCCTGCTGTACCTCTGCAAGCCCAACTGTATGCTTT
TTGAAAGAGAGACTCCAGCTTAAACATTTATCACTTCTCACCACTCTGTC
AAATAGAGTCTGCTCACAATATGCTGCTTATGGGGAGAAGAAATCAAGGC
TCAGCAATCTCATAAAGTTAGCCCAAAAAGTGCCTACTGCTGATCTGGAG
GATGTTTTGCCACTAGCTGAAGATATTACTAACATCCTCTCCAAATGCTG
TGAGTCTGCCTCTGAAGATTGCATGGCCAAAGAGCTGCCTGAACACACAG
TAAAACTCTGTGACAATTTATCCACAAAGAATTCTAAGTTTGAAGACTGT
TGTCAAGAAAAAACAGCCATGGACGTTTTTGTGTGCACTTACTTCATGCC
AGCTGCCCAACTCCCCGAGCTTCCAGATGTAGAGTTGCCCACAAACAAAG
ATGTGTGTGATCCAGGAAACACCAAAGTCATGGATAAGTATACATTTGAA
CTAAGCAGAAGGACTCATCTTCCGGAAGTATTCCTCAGTAAGGTACTTGA
GCCAACCCTAAAAAGCCTTGGTGAATGCTGTGATGTTGAAGACTCAACTA
CCTGTTTTAATGCTAAGGGC
CCTCTACTAAAGAAGGAACTATCTTCTTTCATTGACAAGGGACAAGAACT
ATGTGCAGATTATTCAGAAAATACATTTACTGAGTACAAGAAAAAACTGG
CAGAGCGACTAAAAGCAAAATTGCCTGATGCCACACCCACGGAACTGGCA
AAGCTGGTTAACAAGCACTCAGACTTTGCCTCCAACTGCTGTTCCATAAA
CTCACCTCCTCTTTACTGTGATTCAGAGATTGAGGCTGAATTGACGAATA
TCCTGTAGTCCTGAAGCATGTTTATTAACTTTGACCAGAGTTGGAGCCAC
CCAGGGGAATGATCTCTGATGACCTAACCTAAGCAAAACCACTGAGCTTC
TGGGAAGACAACTAGGATACTTTCTACTTTTTCTAGCTACAATATCTTCA
TACAATGACAAGTATGATGATTTGCTATCAAAATAAATTGAAATATAATG
CAAACCATAAAAAAAAAAAAAAAAAAAAAAA
SEQ ID NO: 7
Primer
artificial DNA sequence
TGTAGTAAGACCTTACATTTAAATGG
SEQ ID NO: 8
Primer
artificial DNA sequence
TACGTTCTTAAAAGATTCTGCCATG

1. A method for determining the susceptibility to, predisposition for, presence of, and/or risk of developing or suffering from endometriosis in a female subject, said method comprising the steps of
(a) obtaining a biological sample from said subject; and
(b) determining the presence of
(i) the vitamin D-binding protein (DBP) GC*2 allele product or a fragment thereof;
(ii) the DBP GC*2 allele or a fragment thereof; or
(iii) a transcription product, preferably an mRNA, of the DBP GC*2 allele, or a fragment of said transcription product
in said sample,
wherein the presence of the DBP GC*2 allele gene product or fragment thereof, the DBP GC*2 allele or fragment thereof, or transcription product of the DBP GC*2 allele or fragment thereof is indicative for an increased susceptibility to, predisposition for and/or risk of developing or suffering from endometriosis and/or for the presence of endometriosis.

2. The method of claim 1, further comprising the step of
(c) determining the presence of
(i) the vitamin D-binding protein (DBP) GC*1F or GC*1S allele product or a fragment thereof;
(ii) the DBP GC*1F or GC*1S allele or a fragment thereof; or
(iii) a transcription product, preferably an mRNA, of the DBP GC*1F or GC*1S allele, or a fragment of said transcription product
in said sample,
wherein
the presence of the DBP GC*2 allele product or fragment thereof, the DBP GC*2 allele or fragment thereof, or transcription product of the DBP GC*2 allele or fragment thereof, and the absence of the DBP GC*1F or GC*1S allele product or fragment thereof, the DBP GC*1F or GC*1S allele or fragment thereof, or transcription product of the DBP GC*1F or GC*1S allele or fragment thereof is indicative for a highly increased susceptibility to, predisposition for and/or risk of developing or suffering from endometriosis and/or for the presence of endometriosis.

3. The method of claim 1, wherein the DBP GC*2 allele or the transcription product thereof comprises a nucleic acid molecule selected from the group consisting of
(a) a nucleic acid molecule comprising SEQ ID NO: 2;
(b) a nucleic acid molecule encoding an amino acid sequence comprising SEQ ID NO: 1; and
(c) a nucleic acid molecule hybridizing under (highly) stringent conditions to a nucleic acid molecule of any one of (a) and (b).

4. The method of claim 1, wherein the DBP GC*2 allele product comprises an amino acid sequence selected from the group consisting of
(a) an amino acid sequence comprising SEQ ID NO: 1;
(b) an amino acid sequence encoded by a nucleic acid molecule of SEQ ID NO: 2; and
(c) an amino acid sequence encoded by a nucleic acid molecule hybridizing under (highly) stringent conditions to a nucleic acid molecule of SEQ ID NO: 2 or to a nucleic acid molecule encoding an amino acid sequence as defined in (a) or (b).

5. The method of claim 2, wherein the DBP GC*1S allele or the transcription product thereof comprises a nucleic acid molecule selected from the group consisting of
(a) a nucleic acid molecule comprising SEQ ID NO: 6;
(b) a nucleic acid molecule encoding an amino acid sequence comprising SEQ ID NO: 3; and
(c) a nucleic acid molecule hybridizing under (highly) stringent conditions to a nucleic acid molecule of any one of (a) and (b).

6. The method of claim 2, wherein the DBP GC*1S allele product comprises an amino acid sequence selected from the group consisting of
(a) an amino acid sequence comprising SEQ ID NO: 3;
(b) an amino acid sequence encoded by a nucleic acid molecule comprising SEQ ID NO: 6; and
(c) an amino acid sequence encoded by a nucleic acid molecule hybridizing under (highly) stringent conditions to a nucleic acid molecule of SEQ ID NO: 6 or to a nucleic acid molecule encoding an amino acid sequence as defined in (a) or (b).

7. The method of claim 2, wherein the DBP GC*1F allele or the transcription product thereof comprises a nucleic acid molecule selected from the group consisting of
(a) a nucleic acid molecule comprising SEQ ID NO: 5;
(b) a nucleic acid molecule encoding an amino acid sequence comprising SEQ ID NO: 4; and
(c) a nucleic acid molecule hybridizing under (highly) stringent conditions to a nucleic acid molecule of any one of (a) and (b).

8. The method of claim 2, wherein the DBP GC*1F allele product comprises an amino acid sequence selected from the group consisting of
(a) an amino acid sequence comprising SEQ ID NO: 4;
(b) an amino acid sequence encoded by a nucleic acid molecule comprising SEQ ID NO: 5; and
(c) an amino acid sequence encoded by a nucleic acid molecule hybridizing under (highly) stringent conditions to a nucleic acid molecule of SEQ ID NO: 5 or to a nucleic acid molecule encoding an amino acid sequence as defined in (a) or (b).

9. The method of claim 1, wherein the subject is human.

10. The method of claim 1, wherein the determination of step (b)(i) is carried out by ELISA.

11. The method of claim 1, wherein the determination of step (c)(i) is carried out by ELISA.

12. The method of claim 1, wherein the determination of step (b)(ii) is carried out by PCR.

13. The method of claim 1, wherein the determination of step (c)(ii) is carried out by PCR.

14. The method of claim 1, wherein the determination of step (b)(iii) is carried out by RT-PCR or Microarray.

15. The method of claim 1, wherein the determination of step (c)(iii) is carried out by RT-PCR or Microarray.

16. The method of claims 1, wherein the biological sample is selected from the group consisting of blood, serum, plasma, blood cells, other blood derived products, saliva, vaginal fluid, urine, and cerebrospinal fluid.

17. The method of claim 16, wherein the biological sample is serum.

18. The method of claim 16, wherein the biological sample is blood cells.

19. The method of claim 1, wherein the GBP GC*1 allele product is glycosylated.

20. A kit useful for determining the risk of developing or suffering from endometriosis and/or the presence of endometriosis in a subject, said kit comprising one or more binding molecules specifically binding to the GC*2 allele product or a fragment thereof, the GC*2 allele or a fragment thereof, and/or the transcription product of a GC*2 allele or a fragment thereof

21. A binding molecule specifically binding to the DBP GC*2 allele product; the DBP GC*1F; or GC*1S allele product.

22. (canceled)

23. The binding molecule of claim 21 which is an antibody.

24. A binding molecule specifically binding to the gene encoding the DBP GC*2 allele product or the DBP GC*1 allele product.

25. (canceled)

26. The binding molecule of claim 24 which is a primer.

27. The binding molecule of claim 24 which is a probe.
